(19) United States
(12) Patent Application Publication
(10) Pub. No.: US 2024/0239871 A1
(43) Pub. Date: Jul. 18, 2024

(54) HYPERSIALYLATING CELLS

(71) Applicant: Novartis AG, Basel (CH)

(72) Inventors: Dominique BREES, Arlesheim (CH); Sebastian FREYSE, Weil am Rhein (DE); Michael KAMMÜLLER, Rheinfelden (DE); Holger LAUX, Marburg (DE); Beat Christoph RAHN, Magden (CH); Babette WOLF, Riehen (CH)

(21) Appl. No.: 18/557,772

(22) PCT Filed: Apr. 27, 2022
(86) PCT No.: PCT/IB2022/053881

§ 371 Date: Oct. 27, 2023

Related U.S. Application Data
(60) Provisional application No. 63/181,739, filed on Apr. 29, 2021.
(60) Provisional application No. 63/181,746, filed on Apr. 29, 2021.

(51) Int. Cl. C07K 16/00; C07K 14/47; C12N 9/10; C12N 15/85

(52) U.S. Cl. CPC C07K 16/00; C07K 14/47; C12N 9/1048; C12N 9/1051; C12N 15/85; C12Y 204/01; C12Y 204/99001; C07K 2317/41; C12N 2800/107

(57) ABSTRACT

The present invention pertains to mammalian cells with increased sialylation activity. The mammalian cells are transfected with coding sequences of a sialyltransferase, a galactosyltransferase and a sialic acid transporter, resulting in hypersialylation of recombinantly expressed glycoproteins.

### FIELD OF THE INVENTION

The present invention pertains to the field of recombinant protein production. Host cells with increased sialylation activity are provided. In particular, a sialyltransferase gene, a galactosyltransferase gene and a sialic acid transporter gene are introduced into a host cell, resulting in hypersialylation of recombinantly expressed glycoproteins. Thereby, proteins with a very high amount of sialic acids are produced.

### BACKGROUND OF THE INVENTION

The Chinese hamster ovary (CHO) cell line is the most widely used mammalian cell line for production of therapeutic proteins and exhibits high productivities in the gram per liter range for antibodies and other therapeutic protein formats. Especially expression of recombinant non-antibody therapeutic proteins is of increasing importance.

It is described in the literature that hypersialylation of therapeutic proteins results in increased drug half-life. Insufficient sialylation (exposure of subterminal galactose) can result in faster clearance of proteins through the asialoglycoprotein receptor-mediated pathways (Bork et al. (2009) Journal of Pharmaceutical Sciences 98:3499-3508). There are several examples of how hypersialylation improves the overall therapeutic efficacies of important biopharmaceutical proteins (Morell et al. (1971) JBC 246(5):1461-7; Richards et al. (2010) Mol Endocrinol 24(1):229-39; Datta-Mannan et al. (2015) Drug Metab Dispos 43:1882-90). These include for example asparaginase, leptin, luteinizing hormone and cholinesterase. Also hypersialylation could result in reduced immunogenicity of non-human therapeutic proteins by shielding antigenic sites.

Antibodies are one example of therapeutic proteins which activities are influenced by the degree of sialylation. Fc-fragments of antibodies possess two conserved N-glycosylation sites on asparagine 297 in the CH2 domain of each heavy chain. Monoclonal antibodies (Mabs) produced in mammalian cells possess a wide variety of glycoforms, as the attached glycans are modified to different extents with core-fucosylation, bisecting N-acetylglucosamine addition, galactosylation and sialylation. The glycan composition is important, as the presence or absence of a single monosaccharide residue can remarkably affect the affinity of the Mab for the different Fcγ-receptors.

Among the variety of monosaccharides present on Fc glycans, terminal sialic acids are particularly interesting. Sialylation of the Fc glycan dramatically decreases Mab affinity for the canonical Fc receptors, thereby inhibiting antibody-dependent cellular cytotoxicity (ADCC), a biological mechanism crucial for the efficacy of several anti-cancer antibodies. Moreover, recent studies on the anti-inflammatory properties of intravenous immunoglobulins (IVIg) suggest that this biological activity could be conferred by the presence of α2,6-sialic acid residues on the Fc glycans (Kaneko et al. (2006) Science 313:670-3; Anthony et al. (2008) Science 320:373-6). Therefore enhanced anti-inflammatory activity may be achieved by producing recombinant IgG therapeutics that contain α2,6-linked sialic acid. Unfortunately, CHO cells lack the enzyme responsible for attaching sialic acids in the α2,6-conformation and only produce glycoproteins with α2,3-linked sialic acids and these only to a low percentages.

In view of the above, there is a need in the art to provide host cells, especially CHO cells, with improved α2,6 sialylation activity.

### SUMMARY OF THE INVENTION

The present inventors have found that overexpression of a sialyltransferase, a galactosyltransferase and a sialic acid transporter together in a host cell dramatically increases the sialylation activity of the cell. Especially using an α2,6-sialyltransferase, a β1,4-galactosyltransferase and a CMP-sialic acid transporter in a CHO cell, the amount of α2,6-linked sialic acids in the produced glycoproteins is significantly enhanced. Introducing the genes of these enzymes via stable transfection with one vector comprising all three coding sequences provides a stable host cell line. Especially good results are obtained with vectors which had the α2,6-sialyltransferase under the control of a strong promoter while expression of the @1,4-galactosyltransferase and the CMP-sialic acid transporter is controlled by a moderate promoter.

As shown by the present inventors, proteins produced in such host cells have a significantly increased amount of sialylation, especially of α2,6-linked sialylation. The present inventors could further demonstrate that antibodies having a respective high level of sialylation have a reduced immunogenicity. Especially, hypersialylation of antibodies reduces their recognition, uptake and presentation by dendritic cells and decreases T cell activation. This transforms into decreased formation of anti-drug antibody since less T helper cells and in turn less B cells are activated. In consequence, increased sialylation of therapeutic antibodies can reduce adverse side effects, in particular side effects caused by immune responses of the patient against the therapeutic antibody.

In view of the above, in a first aspect, the present invention is directed to a mammalian cell which is engineered for increased expression of an alpha-2,6-sialyltransferase, a beta-1,4-galactosyltransferase and a CMP-sialic acid transporter. In certain embodiments, the mammalian cell comprises
(i) an exogenous nucleic acid encoding an alpha-2,6-sialyltransferase;
(ii) an exogenous nucleic acid encoding a beta-1,4-galactosyltransferase; and
(iii) an exogenous nucleic acid encoding a CMP-sialic acid transporter.

In further embodiments, endogenous genes of the mammalian cell encoding an alpha-2,6-sialyltransferase, a beta-1,4-galactosyltransferase and a CMP-sialic acid transporter are engineered for increased expression.

In a second aspect, the present invention provides a method for producing a glycosylated polypeptide, comprising the steps of
(a) providing a mammalian cell according to the first aspect of the present invention which further comprises an expression cassette for recombinant expression of a glycosylated polypeptide;
(b) cultivating the mammalian cell in a cell culture under conditions which allow for the expression of said glycosylated polypeptide;
(c) obtaining said glycosylated polypeptide from the cell culture; and
(d) optionally processing the glycosylated polypeptide.

In certain embodiments of the second aspect, the culture conditions during cultivation of the mammalian cell do not include a temperature shift.

In further embodiments, the method is for producing an antibody or a fragment, derivative or engraft thereof, especially an antibody or a fragment, derivative or engraft thereof with reduced immunogenicity.

In a third aspect, the present invention provides a vector nucleic acid or a combination of at least two vector nucleic acids, comprising
(i) a coding sequence for an alpha-2,6-sialyltransferase;
(ii) a coding sequence for a beta-1,4-galactosyltransferase; and
(iii) a coding sequence for a CMP-sialic acid transporter.

In a fourth aspect, the present invention provides the use of the vector nucleic acid or a combination of at least two vector nucleic acids according to the third aspect of the invention for the transfection of a mammalian cell. The present invention also provides a method for increasing expression of an alpha-2,6-sialyltransferase, a beta-1,4-galactosyltransferase and a CMP-sialic acid transporter in a mammalian cell, comprising the step of transfecting the mammalian cell with the vector nucleic acid or a combination of at least two vector nucleic acids according to the third aspect of the invention, and/or the step of engineering endogenous genes of the mammalian cell encoding an alpha-2,6-sialyltransferase, a beta-1,4-galactosyltransferase and a CMP-sialic acid transporter for increased expression.

In a fifth aspect, the present invention provides a method for reducing the immunogenicity of an antibody or a fragment, derivative or engraft thereof by increasing the amount of sialylation in their glycosylation pattern. The antibody or fragment, derivative or engraft thereof in particular is a therapeutic antibody or fragment, derivative or engraft thereof.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### Definitions

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping. In specific embodiments, a nucleic acid refers to a double-stranded deoxyribonucleic acids.

The term “expression cassette” in particular refers to a nucleic acid construct which is capable of enabling and regulating the expression of a coding nucleic acid sequence introduced therein. An expression cassette may comprise promoters, ribosome binding sites, enhancers and other control elements which regulate transcription of a gene or translation of an mRNA. The exact structure of expression cassette may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences.

According to the invention, the term “promoter” refers to a nucleic acid sequence which is located upstream (5′) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA. The vector according to the present invention may be present in circular or linearized form.

The terms “5′” and “3′” is a convention used to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left hand side, and 3′ (downstream) indicates genetic elements positioned towards the right hand side, when following this convention.

A “polypeptide” refers to a molecule comprising a polymer of amino acids linked together by a peptide bond(s). Polypeptides include polypeptides of any length, including proteins (for example, having more than 50 amino acids) and peptides (for example, having 2-49 amino acids). Polypeptides include proteins and/or peptides of any activity or bioactivity. The polypeptide can be a pharmaceutically or therapeutically active compound, or a research tool to be utilized in assays and the like. Suitable examples are outlined below.

A target amino acid sequence is “derived” from or “corresponds” to a reference amino acid sequence if the target amino acid sequence shares a homology or identity over its entire length with the reference amino acid sequence of at least 75%, more preferably at least 80%, at least 85%, at least 90%, at least 93%, at least 95%, at least 97%, at least 98% or at least 99%. In particular embodiments, a target amino acid sequence which is “derived” from or “corresponds” to a reference amino acid sequence is 100% homologous, or in particular 100% identical, over its entire length with the reference amino acid sequence. Similarly, a target nucleotide sequence is “derived” from or “corresponds” to a reference nucleotide sequence if the target nucleotide sequence shares an identity over its entire length with the reference nucleotide sequence of at least 75%, more preferably at least 80%, at least 85%, at least 90%, at least 93%, at least 95%, at least 97%, at least 98% or at least 99%. In particular embodiments, a target nucleotide sequence which is “derived” from or “corresponds” to a reference nucleotide sequence is 100% identical over its entire length with the reference nucleotide sequence. A “homology” or “identity” of an amino acid sequence or nucleotide sequence is preferably determined according to the invention over the entire length of the reference sequence.

The term “antibody” in particular refers to a protein comprising at least two heavy chains and two light chains connected by disulfide bonds. Each heavy chain is comprised of a heavy chain variable region (V_{H}) and a heavy chain constant region (C_{H}). Each light chain is comprised of a light chain variable region (V_{L}) and a light chain constant region (C_{L}). The heavy chain-constant region comprises three or—in the case of antibodies of the IgM- or IgE-type—four heavy chain-constant domains (C_{H1}, C_{H2}, C_{H3} and C_{H4}) wherein the first constant domain C_{H1} is adjacent to the variable region and may be connected to the second constant domain C_{H2} by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. Preferably, the light chain of the antibody is a κ-chain. The terms “γ- (δ-, α-, μ- or ε-) type heavy chain” and “κ- (λ-) type light chain” refer to antibody heavy chains or antibody light chains, respectively, which have constant region amino acid sequences derived from naturally occurring heavy or light chain constant region amino acid sequences, especially human heavy or light chain constant region amino acid sequences. The antibody can be e.g. a humanized, human or chimeric antibody.

The term “antibody” as used herein also includes fragments, derivatives and engrafts of said antibody. A “fragment or derivative” of an antibody in particular is a protein or glycoprotein which is derived from said antibody and is capable of binding to the same antigen, in particular to the same epitope as the antibody. In further embodiments, a “fragment, derivative or engraft” of an antibody especially refers to polypeptides or proteins which comprise one or more Fc regions of an antibody, and may or may not comprise an antigen binding region. Thus, a fragment, derivative or engraft of an antibody herein generally refers to a functional fragment, derivative or engraft, where the function of the antibody is binding of an antigen and/or interaction with Fc receptors. An “engraft” of an antibody especially refers to said antibody wherein a heterologous polypeptide is introduced into or (partially) replaces a CDR sequence of the antibody. An exemplary antibody engraft is described in US 2017/0158747 A1. In specific embodiments, the antibody or fragment, derivative or engraft thereof comprises a CH2 domain with an N-glycosylation site including the asparagine residue at amino acid position 297 of the antibody heavy chain according to the Kabat numbering.

The term “glycosylated polypeptide” refers to a polypeptide which carries a carbohydrate chain attached to its polypeptide backbone. The carbohydrate chain in particular is attached to the polypeptide by the cellular glycosylation machinery. The carbohydrate chain especially is attached to a glycosylation site of the polypeptide. The term “glycosylation site” in particular refers to an amino acid sequence which can specifically be recognized and glycosylated by a natural glycosylation enzyme, in particular a glycosyltransferase, preferably a naturally occurring mammalian glycosyltransferase. The glycosylated polypeptide especially refers to a polypeptide which carries an N- and/or O-glycosylation. N-glycosylation refers to carbohydrate chains attached to an asparagine residue at an N-glycosylation site having the amino acid sequence Asn-Xaa-Ser/Thr/Cys, wherein Xaa is any amino acid residue. Preferably, Xaa is not Pro. O-glycosylation refers to carbohydrate chains attached to a serine, tyrosine, hydroxy-lysine or hydroxy-proline residue. In specific embodiments, the term “glycosylated polypeptide” refers to a polypeptide which carries a carbohydrate chain attached to an N-glycosylation site.

The term “polypeptide”, as used herein, refers in certain embodiments to a population of polypeptides of the same kind. In particular, all polypeptides of the population of the polypeptide exhibit the features used for defining the polypeptide. In certain embodiments, all polypeptides in the population of the polypeptide have the same amino acid sequence. Reference to a specific kind of polypeptide, such as an antibody, in particular refers to a population of this antibody.

The term “sialic acid” in particular refers to any N- or O-substituted derivatives of neuraminic acid. It may refer to both 5-N-acetylneuraminic acid and 5-N-glycolylneuraminic acid, but preferably only refers to 5-N-acetylneuraminic acid. The sialic acid, in particular the 5-N-acetylneuraminic acid preferably is attached to a carbohydrate chain via a α2,3- or α2,6-linkage.

The term “N-glycosylation” refers to all glycans attached to asparagine residues of the polypeptide chain of a protein. These asparagine residues generally are part of N-glycosylation sites having the amino acid sequence Asn-Xaa-Ser/Thr, wherein Xaa may be any amino acid except for proline. Likewise, “N-glycans” are glycans attached to asparagine residues of a polypeptide chain. The terms “glycan”, “glycan structure”, “carbohydrate”, “carbohydrate chain” and “carbohydrate structure” are generally used synonymously herein. N-glycans generally have a common core structure consisting of two N-acetylglucosamine (GlcNAc) residues and three mannose residues, having the structure Manα1,6-(Manα1,3-)Manβ1,4-GlcNAcβ1,4-GLcNAcβ1-Asn with Asn being the asparagine residue of the polypeptide chain. N-glycans are subdivided into three different types, namely complex-type glycans, hybrid-type glycans and high mannose-type glycans.

According to the invention, an “amount of sialylation” of a polypeptide refers to the amount of glycans which comprise at least one sialic acid residue and which are attached to the polypeptide molecules in a population of the polypeptide. The number of all glycans carrying a sialic acid residue and being attached to the polypeptide of interest in the composition is considered. In specific embodiments, the amount of sialylation refers to the relative amount of sialylation. The relative amount of sialylation refers to the percentage or percentage range of glycans attached to the polypeptide molecules in a population of the polypeptide which are sialylated, based on the total number of all glycans attached to the polypeptide molecules in the population of the polypeptide.

The cells referred to herein in particular are host cells. According to the invention, the term “host cell” relates to any cell which can be transformed or transfected with an exogenous nucleic acid. Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell. The term “cell” as used herein refers in certain embodiments to a population of cells of the same kind. In particular, all cells of the population of the cell exhibit the features used for defining the cell, e.g. they are engineered for increased expression of a certain gene and/or they produce a polypeptide of interest.

An “engineered” cell as used herein refers to cell which was altered on purpose to obtain different properties. Engineering of the cell in particular results in an altered expression of one or more genes in the cell. Especially, the genome of the cell was altered, e.g. by introducing further genetic information into the cell as additional plasmid or as part of the chromosomes already present in the cell, and/or by deleting part of the genetic information in the cell. Furthermore, altered expression of a gene may also be achieved by controlling the transcription and/or translate of a gene, for example by altering the chromosome structure, DNA methylation, codon usage, promoter, transcription activators and/or repressors or by introducing interfering nucleic acids such as siRNA. In specific embodiments, engineering refers to genetic engineering.

The term “pharmaceutical composition” or “pharmaceutical formulation” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

The numbers given herein are preferably to be understood as approximate numbers. In particular, the numbers preferably may be up to 10% higher and/or lower, in particular up to 9%, 8%, 7%, 6%, 5%, 4%, 3%, 2% or 1% higher and/or lower.

Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of compositions refers to subject-matter consisting of the respective steps or ingredients. It is preferred to select and combine preferred aspects and embodiments described herein and the specific subject-matter arising from a respective combination of preferred embodiments also belongs to the present disclosure.

### DETAILED DESCRIPTION OF THE INVENTION

The present invention is based on the development of host cells with high sialylation activity, especially CHO cells, for the production of proteins. The present inventors demonstrated that transfection of host cells with expression cassettes coding for a sialyltransferase, a galactosyltransferase and a sialic acid transporter results in host cells which produce proteins with a high amount of sialic acids. Proteins with a high amount of sialylation generally have a higher circulation half-life. Therefore, the host cells are in particular advantageous for production of therapeutic proteins. In addition to this effect, antibodies with increased sialylation were also shown to be less immunogenic as their recognition and uptake by dendritic cells and their ability to induce a T cell response are decreased.

In view of these findings, the present invention in a first aspect provides a mammalian cell which is engineered for increased expression of an alpha-2,6-sialyltransferase, a beta-1,4-galactosyltransferase and a CMP-sialic acid transporter.

In certain embodiments, the mammalian cell comprises
(i) an exogenous nucleic acid encoding an alpha-2,6-sialyltransferase;
(ii) an exogenous nucleic acid encoding a beta-1,4-galactosyltransferase; and
(iii) an exogenous nucleic acid encoding a CMP-sialic acid transporter.

In further embodiments, endogenous genes of the mammalian cell encoding an alpha-2,6-sialyltransferase, a beta-1,4-galactosyltransferase and a CMP-sialic acid transporter are engineered for increased expression.

Alpha-2,6-sialyltransferase, beta-1,4-galactosyltransferase and CMP-sialic acid transporter are collectively referred to herein as “the glycosylation enzymes”, which term also refers to each enzyme individually. Furthermore, the term “exogenous nucleic acids” as used herein refers to the nucleic acids under (i), (ii) and (iii) all together and also to each of these nucleic acids individually and also to a combination of two of these nucleic acids.

The mammalian cell which is engineered for increased expression of an alpha-2,6-sialyltransferase, a beta-1,4-galactosyltransferase and a CMP-sialic acid transporter in particular has a higher expression of the alpha-2,6-sialyltransferase, the beta-1,4-galactosyltransferase and the CMP-sialic acid transporter compared to the same cell which is not engineered for said increased expression. Thus, the present invention provides a mammalian cell which is engineered for increased expression of an alpha-2,6-sialyltransferase, a beta-1,4-galactosyltransferase and a CMP-sialic acid transporter compared to the same cell which is not engineered for said increased expression.

In particular, the increased expression of the glycosylation enzymes results in an increased sialylation activity of the mammalian cell. The mammalian cell has a higher sialylation activity than the parental cell which was not genetically engineered as described herein. Especially, the mammalian cell is capable of producing proteins with a higher amount of sialic acids than the parental cell which was not genetically engineered. “Parental cell” in this respect in particular refers to the mammalian cell according to the invention before it was engineered. It especially refers to the same cell as the mammalian cell according to the invention, which cell was not engineered as described herein. The amount of sialic acids is especially compared between the same proteins produced under the same conditions. Increased expression of a glycosylation enzyme also includes embodiments wherein the respective glycosylation enzyme is expressed in the mammalian cell, but was not expressed in the parental cell.

### Hypersialylating Host Cells

The mammalian cell may be of any cell type and in particular is a cell useful for recombinantly producing proteins. The mammalian cell may in particular be a rodent cell or a human cell. In certain embodiments, the mammalian cell is selected from, but not limited to, the group consisting of cells derived from mice, such as COP, L, C127, Sp2/0, NS-0, NS-1, At20 and NIH3T3; rats, such as PC12, PC12h, GH3, MtT, YB2/0 and Y0; hamsters, such as BHK, CHO and DHFR gene defective CHO; monkeys, such as COS1, COS3, COS7, CV1 and Vero; and humans, such as Hela, HEK-293, CAP, retina-derived PER-C6, cells derived from diploid fibroblasts, myeloma cells and HepG2. In specific embodiments, the mammalian cell is a Chinese hamster ovary (CHO) cell. The mammalian cell may be suitable for suspension cultures and/or adherent cultures, and in particular can be used in suspension cultures.

The mammalian cell is engineered for increased expression of an alpha-2,6-sialyltransferase, a beta-1,4-galactosyltransferase and a CMP-sialic acid transporter. This engineering may increase the expression of the glycosylation enzymes by introduction of exogenous nucleic acids encoding the glycosylation enzymes or by upregulating endogenous nucleic acids encoding the glycosylation enzymes. A combination of the two options is also possible. For example, for some of the glycosylation enzymes an exogenous nucleic acid may be introduced into the cell and for the other glycosylation enzymes an endogenous nucleic acid is upregulated. Furthermore, for some or all of the glycosylation enzymes both options may be used simultaneous.

In specific embodiments, the mammalian cell comprises
(i) an exogenous nucleic acid encoding an alpha-2,6-sialyltransferase;
(ii) an exogenous nucleic acid encoding a beta-1,4-galactosyltransferase; and
(iii) an exogenous nucleic acid encoding a CMP-sialic acid transporter.

The exogenous nucleic acids are artificially introduced into the mammalian cell. In particular, they are introduced by transfection. Transfection in this respect may be transient or stable, and especially stable transfection is used. Hence, in certain embodiments the mammalian cell comprises the exogenous nucleic acids stably integrated into its genome.

In certain embodiments, the mammalian cell comprises one or more exogenous expression cassettes which comprise the exogenous nucleic acids encoding the glycosylation enzymes. In some embodiments, each of the glycosylation enzymes is expressed by a separate expression cassette. In other embodiments, two of the glycosylation enzymes are expressed by the same expression cassette while the third glycosylation enzyme is expressed by a separate expression cassette. For example, the alpha-2,6-sialyltransferase is expressed by a first expression cassette and the beta-1,4-galactosyltransferase and the CMP-sialic acid transporter are both expressed by a second expression cassette. In even further embodiments, all three glycosylation enzymes are expressed by the same expression cassette. In embodiments wherein two or more glycosylation enzymes are expressed by the same expression cassette, the expression cassette may further comprise an internal ribosome entry site (IRES) or a coding sequence for a 2A element between the coding sequences for the different glycosylation enzymes. A 2A element is a polypeptide stretch which is directly fused to the polypeptides of the preceding and the following glycosylation enzyme. The coding sequences are expressed in one single open reading frame and “self-cleavage” occurs co-translationally.

Each exogenous expression cassette in particular comprises a promoter operatively linked to a coding sequence for one of the glycosylation enzymes or operatively linked to coding sequences for two or three of the glycosylation enzymes. In certain embodiments, the mammalian cell comprises
(i) a first exogenous expression cassette comprising a first promoter operatively linked to a coding sequence for an alpha-2,6-sialyltransferase;
(ii) a second exogenous expression cassette comprising a second promoter operatively linked to a coding sequence for a beta-1,4-galactosyltransferase; and
(iii) a third exogenous expression cassette comprising a third promoter operatively linked to a coding sequence for a CMP-sialic acid transporter.

In other embodiments, the mammalian cell comprises
(i) a first exogenous expression cassette comprising a first promoter operatively linked to a coding sequence for an alpha-2,6-sialyltransferase; and
(ii) a second exogenous expression cassette comprising a second promoter operatively linked to a coding sequence for a beta-1,4-galactosyltransferase and to a coding sequence for a CMP-sialic acid transporter.

In these embodiments, the second exogenous expression cassette in particular comprises an IRES between the coding sequence for a beta-1,4-galactosyltransferase and the coding sequence for a CMP-sialic acid transporter.

The expression cassettes typically further comprise an mRNA processing and translational signal which usually includes a Kozak sequence, and an mRNA polyadenylation signal. The elements of an expression cassette which are necessary for enabling expression of the coding sequence are known to the person skilled in the art. The elements of the expression cassette in particular are selected for expression in a mammalian cell.

The promoter used in the expression cassettes may be any promoter suitable for driving expression in a mammalian host cell. The promoter may for example be selected from the group consisting of cytomegalovirus (CMV) promoter, simian virus 40 (SV40) promoter, ubiquitin C (UBC) promoter, elongation factor 1 alpha (EF1A) promoter, phosphoglycerate kinase (PGK) promoter, Rous sarcoma virus (RSV) promoter, BROAD3 promoter, murine rosa 26 promoter, pCEFL promoter and β-actin promoter optionally coupled with CMV early enhancer (CAGG). Specific examples of promoters include cytomegalovirus immediate-early promoter, simian virus 40 early promoter, human Ubiquitin C promoter, human elongation factor 1a promoter, mouse phosphoglycerate kinase 1 promoter, Rous sarcoma virus long terminal repeat promoter and chicken β-Actin promoter coupled with CMV early enhancer.

In certain embodiments, the promoter operatively linked to a coding sequence for the alpha-2,6-sialyltransferase, especially the first promoter of the above embodiments, is a strong promoter. A strong promoter for example is a promoter which effects high expression of the regulated coding sequence. A high expression in this respect refers to an expression which results in an amount of transcripts of the coding sequence which is at least as high as the amount of transcripts of a highly expressing housekeeping gene of the mammalian cell. Suitable examples of highly expressing housekeeping genes are EEF1A1 (eukaryotic translation elongation factor 1 alpha 1 gene), ACTB (beta actin gene) and PPIA (peptidylprolyl isomerase A gene). In certain embodiments, the amount of transcripts of the coding sequence is at least 1.5 times higher or at least 2 times higher than the amount of transcript of a highly expressing housekeeping gene of the mammalian cell. The amount of transcripts may be measured, for example, using next generation sequencing or real-time RT-PCR. The amount of transcripts may be normalized, for example, to fragments per kilobase of transcript per million mapped reads (FPKM), e.g. as described in the examples. Hence, a high expression results in a FPKM value which is at least as high as that of a highly expressing housekeeping gene of the mammalian cell, such as EEF1A1, ACTB and PPIA, in particular at least 1.5 times higher, especially at least 2 times higher. In certain embodiments, the first promoter is a strong promoter which effects high expression of the coding sequence for an alpha-2,6-sialyltransferase resulting in an amount of transcripts of the coding sequence which is at least as high as the amount of transcripts of at least one of EEF1A1, ACTB and PPIA in the mammalian cell.

Suitable promotors operatively linked to a coding sequence for the alpha-2,6-sialyltransferase may be selected from the group consisting of CMV promoter, EF1alpha promoter, RSV promoter, BROAD3 promoter, murine rosa 26 promoter, pCEFL promoter and β-actin promoter. In specific embodiments, the promoter operatively linked to a coding sequence for the alpha-2,6-sialyltransferase, especially the first promoter of the above embodiments, is a CMV promoter.

In certain embodiments, the promoter operatively linked to a coding sequence for the beta-1,4-galactosyltransferase and/or to a coding sequence for the CMP-sialic acid transporter, especially the second and/or third promoter of the above embodiments, is a moderate promoter. Suitable promotors in this case may be selected from the group consisting of SV40 promoter, CMV promoter, UBC promoter, EF1A promoter, PGK promoter and CAGG promoter. In specific embodiments, the promoter operatively linked to a coding sequence for the beta-1,4-galactosyltransferase and/or to a coding sequence for the CMP-sialic acid transporter is a SV40 promoter. In certain embodiments, the promoter operatively linked to a coding sequence for the beta-1,4-galactosyltransferase and/or to a coding sequence for the CMP-sialic acid transporter, especially the second and/or third promoter of the above embodiments, is selected from the group consisting of simian virus 40 (SV40) promoter, CMV promoter, ubiquitin C (UBC) promoter, elongation factor 1 alpha (EF1A) promoter, phosphoglycerate kinase (PGK) promoter and β-actin promoter coupled with CMV early enhancer (CAGG), in particular SV40 promoter.

In specific embodiments, the promoter operatively linked to a coding sequence for the alpha-2,6-sialyltransferase, especially the first promoter of the above embodiments, effects a higher expression than the promoter(s) operatively linked to a coding sequence for the beta-1,4-galactosyltransferase and/or to a coding sequence for the CMP-sialic acid transporter, especially the second and/or, if present, third promoter of the above embodiments. A higher expression in this respect refers to an expression which results in a higher amount of transcripts of the coding sequence for the alpha-2,6-sialyltransferase compared to the amount of transcripts of the coding sequence for the beta-1,4-galactosyltransferase and/or the CMP-sialic acid transporter. In particular, the expression and/or the amount of transcripts is at least 1.5 times higher, especially at least 2 times, at least 3 times, at least 5 times or at least 10 times higher. The amount of transcripts may be measured, for example, using next generation sequencing. The amount of transcripts may be normalized, for example, to fragments per kilobase of transcript per million mapped reads (FPKM), e.g. as described in the examples. Hence, a higher expression results in a higher FPKM value, in particular in an at least 1.5 times higher, especially at least 2 times, at least 3 times, at least 5 times or at least 10 times higher FPKM value. In general, a strong promoter in particular results in a higher FPKM value of the controlled coding sequence compared to a moderate promoter.

In further embodiments, endogenous genes of the mammalian cell encoding an alpha-2,6-sialyltransferase, a beta-1,4-galactosyltransferase and a CMP-sialic acid transporter are engineered for increased expression. In these embodiments, expression of the endogenous glycosylation enzyme genes that are present in the host cell genome is upregulated to achieve increased expression of the glycosylation enzymes. In these embodiments, the mammalian cell may or may not, and especially does not comprise exogenous nucleic acids encoding the glycosylation enzymes. Upregulating of an endogenous gene results in a higher expression of said gene. Upregulating also includes activating a gene which was not expressed before engineering of the cell.

Different technologies to modulate expression of endogenous genes are known and can be applied to increase transcription levels of alpha-2,6-sialyltransferase, beta-1,4-galactosyltransferase, and CMP-sialic acid transporter. Non-limiting examples of technologies to upregulate gene expression include ZFN-activators, TALEN-activators, and CRISPR-activators which can be designed to selectively bind to the promoter regions of the genes of the glycosylation enzymes and upon binding act like transcription factors to activate gene expression. Alternatively, promoter regions of the glycosylation enzymes of the host cell can be engineered to insert one or more promoter and/or enhancer elements leading to an increase in expression levels. In further embodiments, chromatin modulation entities are used that change conformation of the chromatin structure around the loci of the glycosylation enzymes towards a transcriptionally more active state. Non-limiting examples of such entities include matrix attachment regions (MARs), ubiquitous chromatin opening elements (UCOEs), STAR elements and comparable sequence motifs. Similar chromatin modulation capabilities have also been described for proxy-CRISPR-based entities which also can serve the purpose to increase expression of the glycosylation enzymes. In further embodiments, codon modification is used to increase translation of the glycosylation enzymes of the host cell. These technologies can also be used in combination with each other.

In specific embodiments, a promoter is introduced into an endogenous gene of the respective glycosylation enzyme so that it is operatively linked to the coding sequence for said glycosylation enzyme. In certain embodiments, the mammalian cell comprises
(i) a first exogenous promoter operatively linked to an endogenous coding sequence for an alpha-2,6-sialyltransferase;
(ii) a second exogenous promoter operatively linked to an endogenous coding sequence for a beta-1,4-galactosyltransferase;
(i) a third exogenous promoter operatively linked to an endogenous coding sequence for a CMP-sialic acid transporter.

Suitable promoters for the different glycosylation enzymes are described herein. Especially, the same promoters as used for the exogenous nucleic acids may also be used for the endogenous nucleic acids. Alternatively or additionally, an enhancer may be introduced into the endogenous gene of one or more of the glycosylation enzymes. This enhancer may increase the activity of the endogenous promotor of the glycosylation enzyme gene. Introduction of the promoters and/or enhancers into the genome of the mammalian cell may be done by any known method for genetic engineering. An exemplary method is the use of the CRISPR technology.

In specific embodiments, exogenous nucleic acids encoding the glycosylation enzymes and increasing the expression of endogenous nucleic acids encoding the glycosylation enzymes may be combined. For example, for one or two of the glycosylation enzymes a respective exogenous nucleic acid is introduced into the mammalian cells while for the remaining two or one glycosylation enzymes the expression of the respective endogenous nucleic acid is increased. In further embodiments, for one, two or all three of the glycosylation enzymes, both a respective exogenous nucleic acid is introduced into the mammalian cells and the expression of the respective endogenous nucleic acid is increased.

The alpha-2,6-sialyltransferase may be any enzyme which is capable of effecting attachment of a sialic acid residue via an α2,6-linkage to a terminal galactose residue of a complex-type N-linked oligosaccharide in a mammalian cell. In certain embodiments, the alpha-2,6-sialyltransferase is derived from Cricetulus griseus or human. In particular, the alpha-2,6-sialyltransferase is beta-galactoside alpha-2,6-sialyltransferase 1 (ST6GAL1), especially from Cricetulus griseus or human. In some embodiments, the alpha-2,6-sialyltransferase has an amino acid sequence derived from accession number P15907 of the UniProt database or from the amino acid sequence of SEQ ID NO: 1 or 2.

The beta-1,4-galactosyltransferase may be any enzyme which is capable of effecting attachment of a galactose residue via a β1,4-linkage to a terminal GlcNAc residue of a complex-type N-linked oligosaccharide in a mammalian cell. In certain embodiments, the beta-1,4-galactosyltransferase is derived from Cricetulus griseus or human. In particular, the beta-1,4-galactosyltransferase is beta-1,4-galactosyltransferase 1 (B4GALT1), especially from Cricetulus griseus or human. In some embodiments, the alpha-2,6-sialyltransferase has an amino acid sequence derived from accession number P15291 of the UniProt database or from the amino acid sequence of SEQ ID NO: 3 or 4.

The CMP-sialic acid transporter may be any enzyme which is capable of transporting a CMP-sialic acid residue into the Golgi of a mammalian cell. In certain embodiments, the CMP-sialic acid transporter is derived from Cricetulus griseus or human. In particular, the CMP-sialic acid transporter is CMP-sialic acid transporter (SLC35A1), especially from Cricetulus griseus or human. In some embodiments, the alpha-2,6-sialyltransferase has an amino acid sequence derived from accession number 008520 or P78382 of the UniProt database or from the amino acid sequence of SEQ ID NO: 5 or 6.

In certain embodiments, the glycosylation enzymes are derived from the same species, especially the same species as the mammalian cell, in particular from Cricetulus griseus or human. For example, in case of a Chinese hamster cell such as CHO, the glycosylation enzymes are derived from Cricetulus griseus. In case a human cell is used, the glycosylation enzymes are derived from human. In other embodiments, the glycosylation enzymes are derived from Cricetulus griseus while the mammalian cell is not a hamster cell, but for example a human cell; or the glycosylation enzymes are derived from human while the mammalian cell is not a human cell, but for example a CHO cell. In further embodiments, the glycosylation enzymes may also be derived from other species, especially from other mammals, in particular rodents such as mouse and rat, especially Mus musculus and Rattus norvegicus.

In certain embodiments, the exogenous nucleic acids encoding the glycosylation enzymes are present on a vector or a combination of two or three vectors used for transformation of the mammalian cell. In particular, the mammalian cell was obtained by transformation with a vector or a combination of two or three vectors comprising the exogenous nucleic acids. In specific embodiments, one vector comprising all three exogenous nucleic acids is used. In particular, the vector or the combination of two or three vectors comprises the exogenous expression cassette as described herein.

In some embodiments, each vector further comprises at least one selectable marker gene. The selectable marker gene in particular is a mammalian selectable marker gene which allows the selection of mammalian host cells comprising said gene and thus of mammalian host cells comprising the vector.

Non-limiting examples of mammalian selectable marker genes include antibiotic resistance genes e.g. conferring resistance to G418; hygromycin (hyg or hph, commercially available from Life Technologies, Inc. Gaithesboro, Md.); neomycin (neo, commercially available from Life Technologies, Inc. Gaithesboro, Md.); zeocin (Sh Ble, commercially available from Pharmingen, San Diego Calif.); puromycin (pac, puromycin-N-acetyl-transferase, available from Clontech, Palo Alto Calif.), ouabain (oua, available from Pharmingen) and blasticidin (available from Invitrogen). Further suitable selectable marker genes include folate receptor genes such as the folate receptor alpha gene, or genes encoding fluorescent proteins such as GFP and RFP. Respective mammalian selectable marker genes are well known and allow the selection of mammalian cells comprising said genes and thus of cells comprising the vector. Systems using a folate receptor gene are described in WO 2009/080759 and WO 2015/015419. The term “gene” as used herein also refers to a natural or synthetic polynucleotide encoding a functional variant of the selectable marker providing the intended resistance. Hence, also truncated or mutated versions of a wild type gene or synthetic polynucleotides are encompassed as long as they provide the intended resistance. According to a specific embodiment, the vector comprises a gene encoding an enzymatically functional puromycin-N-acetyl-transferase (pac) as selectable marker gene.

In some embodiments, the mammalian selectable marker genes may be amplifiable and allow selection of vector-containing mammalian host cells as well as gene amplification. A non-limiting example for an amplifiable, selectable mammalian marker gene is the dihydrofolate reductase (DHFR) gene. Other systems currently in use are among others the glutamine synthetase (gs) system and the histidinol driven selection system. These amplifiable markers are also selectable markers and can thus be used to select those cells that obtained the vector. With amplifiable systems such as the DHFR system, expression of a recombinant protein can be increased by exposing the cells to certain agents promoting gene amplification such as antifolates (e.g. methotrexate (MTX)) in case of the DHFR system. A suitable inhibitor for GS promoting gene amplification is methionine sulphoximine (MSX). Exposure to MSX also results in gene amplification.

The selectable marker gene may be positioned on the vector upstream of, downstream of or in between the expression cassette(s) for the glycosylation enzymes. In certain embodiments, the selectable marker gene is positioned on the vector downstream of the expression cassette(s) for the glycosylation enzymes. In specific embodiments, the vector comprises a second, different selectable marker gene. In these embodiments, preferably one selectable marker gene is positioned on the vector downstream of the expression cassette(s) for the glycosylation enzymes and the other selectable marker gene is positioned on the vector upstream of the expression cassette(s) for the glycosylation enzymes. In embodiments wherein a combination of two or three vectors is used, the different vectors in particular comprise different selectable marker genes.

The vector or combination of vectors in particular is suitable for integration into the genome of the mammalian cell. In some embodiments, the mammalian cell is stably transfected with the exogenous nucleic acids. In certain embodiments, the vector further comprises a prokaryotic selectable marker gene. Said prokaryotic selectable marker may provide a resistance to antibiotics such as e.g. ampicillin, kanamycin, tetracycline and/or chloramphenicol.

In certain embodiments, the mammalian cell further comprises an exogenous expression cassette for recombinant expression of a glycosylated polypeptide. The exogenous expression cassette for recombinant expression of a polypeptide generally is introduced into the mammalian cell separately from the exogenous nucleic acids encoding the glycosylation enzymes. For example, the mammalian cell is transfected with a further vector comprising the exogenous expression cassette for recombinant expression of a glycosylated polypeptide. The glycosylated polypeptide may be any glycosylated polypeptide of interest, especially including hormones, cytokines, enzymes, antibodies, fusion proteins, vaccines, coagulation proteins, toxins and growth factors. In certain embodiments, the glycosylated polypeptide is selected from the group consisting of antibodies and fragments, derivatives or engrafts thereof, in particular proteins comprising an antibody Fc region, whole antibodies, and Fc multimers comprising two or more antibody Fc regions. In certain embodiments, the glycosylated polypeptide is a pharmaceutically active polypeptide, such as a therapeutic polypeptide or diagnostic polypeptide, especially a therapeutic antibody, therapeutic antibody fragment, therapeutic antibody derivative or therapeutic antibody engraft.

### The Method for Producing a Glycosylated Polypeptide

In a second aspect, the present invention provides a method for producing a glycosylated polypeptide, comprising the steps of
(a) providing a mammalian cell according to the first aspect of the present invention which further comprises an expression cassette for recombinant expression of a glycosylated polypeptide;
(b) cultivating the mammalian cell in a cell culture under conditions which allow for the expression of said glycosylated polypeptide;
(c) obtaining said glycosylated polypeptide from the cell culture; and
(d) optionally processing the glycosylated polypeptide.

The embodiments, features and examples described herein with respect to the mammalian cell likewise apply to the method for producing a glycosylated polypeptide using such a mammalian cell.

In certain embodiments, the method further comprises between steps (a) and (b) the steps of
(a1) inoculating a cell culture medium with the mammalian cell to provide a cell culture, and
(a2) cultivating the mammalian cell in the cell culture under conditions which allow for increasing the number of cells in the cell culture.

Suitable conditions for cultivating the mammalian cells, increasing their cell number and expressing the glycosylated polypeptide depend on the specific mammalian cell, vector and expression cassette used in the method. The skilled person can readily determine suitable conditions and they are also already known in the art for a plurality of mammalian cells. In certain embodiments, the mammalian cell is transfected with one or more vectors comprising selectable marker genes. In these embodiments, the culturing conditions in step (a2) and/or (b) may include the presence of a corresponding selection agent in the cell culture medium.

In cultivation methods used in the art, especially when cultivating CHO cells, a temperature shift is performed after the first exponential cell growth phase is finished, e.g. when a desired cell density is reached. For example, the cultivation temperature is lowered from about 37° C. to about 33° C. when reaching a cell density of about 10⁶ cells/ml. However, the present inventors found that keeping the temperature constant, especially at about 36.5° C., a higher viable cell density, a higher product concentration and a higher sialylation level are achieved. Furthermore, due to the omission of a further process event (reduction of the temperature), the entire process is more robust and less likely to experience deviations. Therefore, in specific embodiments, the method does not comprise a temperature shift during cultivation. According to the present invention, a temperature shift refers to a change, especially a reduction, in the temperature of the cell culture by more than 3° C. for a duration of at least 1 hour. In particular, small and/or short time fluctuations in the temperature of the cell culture are not considered to be a temperature shift. In certain embodiments, the culture conditions during cultivation of the mammalian cell do not include a temperature shift of more than 2° C. In specific embodiments, the culture conditions during cultivation of the mammalian cell do not include a temperature shift of more than 1.5° C. In specific embodiments, the culture conditions during cultivation of the mammalian cell do not include a temperature shift of more than 1° C.

In certain embodiments, the method does not comprise a temperature set-point shift during cultivation. The temperature set-point is a predefined, exact temperature value which a control system is aiming to reach. Fluctuations of the measured value which are caused by technical control limitations are possible and are not considered as changes of the temperature set-point. Thus, in these embodiments the temperature set-point is not changed during cultivation or is not changed by more than 2° C., preferably more than 1.5° C., more preferably more than 1° C.

In particular, the temperature is kept within a certain range during the cultivation of the mammalian cells. In particular, this refers to a variation in temperature of less than 2° C. In certain embodiments, the temperature is not reduced or altered by more than 2° C., especially not more than 1.5° C. or not more than 1° C., during cultivation of the mammalian cell. For example, the temperature is kept in the range of from 30° ° C. to 40° C., especially from 32° C. to 39° C. or from 34° C. to 39° C., in particular from 35° ° C. to 38° C., such as at about 36.5° C. In certain embodiments, the temperature is kept within the range of 35° C. to 38° C. during cultivation of the mammalian cell. In specific embodiments, the temperature is kept at 35° C. or more during cultivation of the mammalian cell. In specific embodiments, a deviation from the desired temperature outside of the defined range is allowed if the duration of the deviation is less than 1 hour, especially less than 30 minutes.

Obtaining the glycosylated polypeptide from the cell culture in particular includes isolating the glycosylated polypeptide from the cell culture. Isolation of the glycosylated polypeptide in particular refers to the separation of the glycosylated polypeptide from the remaining components of the cell culture. The term “cell culture” as used herein in particular includes the cell culture medium and the cells. In certain embodiments, the glycosylated polypeptide is secreted by the mammalian cell. In these embodiments, the glycosylated polypeptide is isolated from the cell culture medium. For example, the coding region of the expression cassette for recombinant expression of the glycosylated polypeptide may further comprises a nucleic acid sequence coding for a signal peptide for secretory expression. Separation of the glycosylated polypeptide from the cell culture medium may be performed, for example, by chromatographic methods. Suitable methods and means for isolating the polypeptide of interest are known in the art and can be readily applied by the skilled person.

The obtained glycosylated polypeptide may optionally be subject to further processing steps such as e.g. further purification, modification and/or formulation steps in order to produce the product of interest in the desired quality and composition. Such further processing steps and methods are generally known in the art. Suitable purification steps for example include affinity chromatography, size exclusion chromatography, anion- and/or cation exchange chromatography, hydrophilic interaction chromatography and reverse phase chromatography. Further steps may include virus inactivation, ultrafiltration and diafiltration. Modification steps may include chemical and enzymatic modification reactions such as coupling of chemical entities to the glycosylated polypeptide and enzymatic cleavage of the glycosylated polypeptide. Formulation steps may include buffer exchange, addition of formulation components, pH adjustment, and concentration adjustment. Any combination of these and further steps may be used.

In certain embodiments, the method for producing a glycosylated polypeptide further comprises as step (d) or part of step (d) the step of providing a pharmaceutical formulation comprising the glycosylated polypeptide. Providing a pharmaceutical formulation comprising the glycosylated polypeptide or formulating the glycosylated polypeptide as a pharmaceutical composition in particular comprises exchanging the buffer solution or buffer solution components of the composition comprising the glycosylated polypeptide. Furthermore, this step may include lyophilization of the glycosylated polypeptide. In particular, the glycosylated polypeptide is transferred into a composition only comprising pharmaceutically acceptable ingredients.

In certain embodiments, the glycosylated polypeptide produced in the method has a higher amount of sialic acids compared to the same polypeptide produced in a reference cell under the same conditions, wherein the reference cell is the same as the host cell except that it was not engineered as described herein. In specific embodiments, the method is for producing a glycosylated polypeptide having a higher amount of sialic acids compared to the same polypeptide produced in a reference cell under the same conditions, wherein the reference cell is the same as the host cell except that it was not engineered for increased expression of an alpha-2,6-sialyltransferase, a beta-1,4-galactosyltransferase and a CMP-sialic acid transporter. In particular, the reference cell was not engineered for increased expression of any one of an alpha-2,6-sialyltransferase, a beta-1,4-galactosyltransferase and a CMP-sialic acid transporter.

In certain embodiments, the amount of sialic acids of the glycosylated polypeptide is at least 10 percent points higher than the amount of sialic acids of the same polypeptide produced in a reference cell. The amount of sialic acids is preferably at least 20 percent points higher, more preferably at least 30 percent points higher, and most preferably at least 40 percent points higher.

In further embodiments, the method is for producing an antibody or a fragment, derivative or engraft thereof, especially an antibody or a fragment, derivative or engraft thereof with reduced immunogenicity.

Hence, in a specific embodiment the present invention provides a method for producing a glycosylated polypeptide with reduced immunogenicity, comprising the steps of
(a) providing a mammalian cell according to the first aspect of the present invention which further comprises an expression cassette for recombinant expression of a glycosylated polypeptide;
(b) cultivating the mammalian cell in a cell culture under conditions which allow for the expression of said glycosylated polypeptide;
(c) obtaining said glycosylated polypeptide from the cell culture; and
(d) optionally processing the glycosylated polypeptide;
   wherein the glycosylated polypeptide is an antibody or a fragment, derivative or engraft thereof.

In particular, the glycosylated polypeptide has a reduced immunogenicity compared to a reference polypeptide. The term “reduced immunogenicity” in this respect means that the glycosylated polypeptide is less likely to cause an immune reaction directed against it when administered to a patient compared to a reference polypeptide.

The reference polypeptide has the same amino acid sequence as the glycosylated polypeptide with reduced immunogenicity, but a lower amount of sialylation. In certain embodiments, the reference polypeptide has the same amino acid sequence as the glycosylated polypeptide with reduced immunogenicity, but does not have any sialylation. Alternatively, 5% or less, in particular 1% or less of the carbohydrate structures attached to the reference polypeptide are sialylated. Thus, the reference polypeptide in particular has an amount of sialylation of 5% or less, preferably of 1% or less. In certain embodiments, the amount of sialylation of the glycosylated polypeptide is at least 10 percent points higher than the amount of sialylation of the reference polypeptide. The amount of sialylation is preferably at least 20 percent points higher, more preferably at least 30 percent points higher, and most preferably at least 40 percent points higher.

In particular, the reference polypeptide is produced in a reference cell under the same conditions as the glycosylated polypeptide with reduced immunogenicity, except that the reference cell was not engineered for increased expression of an alpha-2,6-sialyltransferase, a beta-1,4-galactosyltransferase and a CMP-sialic acid transporter. In particular, the reference cell was not engineered for increased expression of any one of an alpha-2,6-sialyltransferase, a beta-1,4-galactosyltransferase and a CMP-sialic acid transporter.

### The Nucleic Acid Coding for the Glycosylation Enzymes

In a third aspect, the present invention provides a vector nucleic acid or a combination of at least two vector nucleic acids, comprising
(i) a coding sequence for an alpha-2,6-sialyltransferase;
(ii) a coding sequence for a beta-1,4-galactosyltransferase; and
(iii) a coding sequence for a CMP-sialic acid transporter.

The coding sequences in particular are part of one or more expression cassettes, wherein each expression cassette comprises a promoter which is operatively linked to the coding sequence(s), and wherein the expression cassettes are for expression in a mammalian host cell.

The embodiments, features and examples described herein with respect to the mammalian cell and the exogenous nucleic acids comprised therein likewise apply to the vector nucleic acid or combination of at least two vector nucleic acids.

In particular, each coding sequence may be part of a separate expression cassette or two or three of the coding sequences may be part of the same expression cassette. For example, the vector nucleic acid or combination of at least two vector nucleic acids may comprise
(i) a first expression cassette comprising a first promoter operatively linked to the coding sequence for an alpha-2,6-sialyltransferase;
(ii) a second expression cassette comprising a second promoter operatively linked to the coding sequence for a beta-1,4-galactosyltransferase; and
(iii) a third expression cassette comprising a third promoter operatively linked to the coding sequence for a CMP-sialic acid transporter.

In another example, the vector nucleic acid or combination of at least two vector nucleic acids may comprise
(i) a first expression cassette comprising a first promoter operatively linked to the coding sequence for an alpha-2,6-sialyltransferase; and
(ii) a second expression cassette comprising a second promoter operatively linked to the coding sequence for a beta-1,4-galactosyltransferase and to the coding sequence for a CMP-sialic acid transporter.

In embodiments wherein an expression cassette comprises two or more coding sequences, the expression cassette may further comprise an IRES or a coding sequence for a 2A element between the coding sequences.

In certain embodiments, the first promoter effects a higher expression than the second promoter and/or the third promoter, if present. In particular, promoters as described above with respect to the expression cassettes used in the mammalian cell as also used for the expression cassettes of the nucleic acid or combination of nucleic acids. In certain embodiments, the first promoter is a cytomegalovirus promoter (CMV). In certain embodiments, the second and/or the third promoter is selected from the group consisting of simian virus 40 promoter (SV40), CMV promoter, ubiquitin C (UBC) promoter, elongation factor 1 alpha (EF1A) promoter, phosphoglycerate kinase (PGK) promoter and β-actin promoter coupled with CMV early enhancer (CAGG), in particular SV40 promoter. In some embodiments, the expression cassettes comprise further elements as described herein, especially a polyadenylation signal.

The glycosylation enzymes in particular are as described herein. In specific embodiments, the alpha-2,6-sialyltransferase is beta-galactoside alpha-2,6-sialyltransferase 1 (ST6GAL1), in particular derived from Cricetulus griseus or human. In specific embodiments, the beta-1,4-galactosyltransferase is beta-1,4-galactosyltransferase 1 (B4GALT1), in particular derived from Cricetulus griseus or human. In specific embodiments, the CMP-sialic acid transporter is CMP-sialic acid transporter (SLC35A1), in particular derived from Cricetulus griseus or human. In certain embodiments, the alpha-2,6-sialyltransferase is beta-galactoside alpha-2,6-sialyltransferase 1 (ST6GAL1) of Cricetulus griseus, the beta-1,4-galactosyltransferase is beta-1,4-galactosyltransferase 1 (B4GALT1) of Cricetulus griseus, and the CMP-sialic acid transporter is CMP-sialic acid transporter (SLC35A1) of Cricetulus griseus.

The different expression cassettes may be present on the same nucleic acid or they may be present on separate nucleic acids, which together form the combination of nucleic acids. In specific embodiments, one nucleic acid comprises all coding sequences for the glycosylation enzymes. In particular, one nucleic acid comprises the expression cassettes as described herein.

In some embodiments, the vector nucleic acid or each vector nucleic acid of the combination of at least two vector nucleic acids further comprises at least one selectable marker gene. Suitable selectable marker genes are described above with respect to the vector used for transforming the mammalian cell. In certain embodiments, the selectable marker gene is an antibiotic resistance gene such as puromycin-N-acetyltransferase gene (pac). In specific embodiments, the vector nucleic acid comprises one selectable marker gene which is positioned on the nucleic acid downstream of the expression cassettes, and optionally a second selectable marker, which is positioned on the nucleic acid upstream of the expression cassettes.

In some embodiments, the vector nucleic acid or a combination of at least two vector nucleic acids is suitable for stable transfection of a host cell, especially a mammalian host cell such as a rodent or human cell, especially a CHO cell. In particular, the vector nucleic acid is a plasmid.

The present invention further provides in a fourth aspect the use of the vector nucleic acid or combination of at least two vector nucleic acids for the transfection of a mammalian cell. In particular, the mammalian cell is a Chinese hamster ovary (CHO) cell.

The present invention also provides a method for increasing expression of an alpha-2,6-sialyltransferase, a beta-1,4-galactosyltransferase and a CMP-sialic acid transporter in a mammalian cell, comprising the step of transfecting the mammalian cell with the vector nucleic acid or a combination of at least two vector nucleic acids as described herein, and/or the step of engineering endogenous genes of the mammalian cell encoding an alpha-2,6-sialyltransferase, a beta-1,4-galactosyltransferase and a CMP-sialic acid transporter for increased expression as described herein.

The embodiments, features and examples described herein with respect to the mammalian cell and the exogenous nucleic acids comprised therein, and the vector nucleic acid or combination of at least two vector nucleic acids likewise apply to the use of the vector nucleic acid or combination of at least two vector nucleic acids for the transfection of a mammalian cell and to the method for increasing expression of an alpha-2,6-sialyltransferase, a beta-1,4-galactosyltransferase and a CMP-sialic acid transporter in a mammalian cell.

### The Method for Reducing the Immunogenicity of Antibodies

In a fifth aspect, the present invention provides a method for reducing the immunogenicity of an antibody or a fragment, derivative or engraft thereof, comprising the step of increasing the amount of sialylation in the glycosylation pattern of the antibody or fragment, derivative or engraft thereof.

The antibody or fragment, derivative or engraft thereof in particular is a therapeutic antibody or fragment, derivative or engraft thereof, preferably a therapeutic antibody. In certain embodiments, the antibody or fragment, derivative or engraft thereof comprises a CH2 domain with an N-glycosylation site including the asparagine residue at amino acid position 297 of the antibody heavy chain according to the Kabat numbering. In certain embodiments, the step of increasing the amount of sialylation in the glycosylation pattern of the antibody or fragment, derivative or engraft thereof includes increasing the amount of sialylation in the N-glycosylation pattern of the CH2 domain.

As a result of the method, the antibody or fragment, derivative or engraft thereof has a higher amount of sialylation.

The method may include a direct increase of the amount of sialylation of the antibody or fragment, derivative or engraft thereof. In this case, a composition comprising the antibody or fragment, derivative or engraft thereof is treated so that sialic acid residues are attached to the glycans present on the antibody or fragment, derivative or engraft thereof. Suitable means for directly increasing the amount of sialylation include, for example, in vitro treatment of the antibody or fragment, derivative or engraft thereof with a sialyltransferase such as alpha-2,6-sialyltransferase as described herein, and a sialic acid donor. The sialyltransferase transfers sialic acid residues to the glycans of a polypeptide and thereby increases the amount of sialylation of the polypeptide.

In further embodiments, the method includes an increase of the amount of sialylation of the antibody or fragment, derivative or engraft thereof by enrichment of those antibody or fragment, derivative or engraft thereof which carry at least one sialic acid. In the resulting composition of the antibody or fragment, derivative or engraft thereof, the relative amount of sialylation is higher than in the composition prior to enrichment. Enrichment of the antibody or fragment, derivative or engraft thereof which carry at least one sialic acid may be achieved by any suitable means.

Exemplary means for enrichment are chromatographic methods such as affinity chromatography using ligands specifically binding to sialylated glycan structures, e.g. lectins or antibodies specific for sialylated glycan structures. A suitable lectin in this respect is for example Sambucus nigra lectin. In these embodiments, sialylated polypeptides are bound to the chromatography matrix and non-sialylated polypeptides are washed away. After elution of the bound polypeptides, the amount of sialylation is increased.

Further means for enrichment include affinity chromatography using ligands specifically binding to non-sialylated glycan structures, e.g. lectins or antibodies specific for non-sialylated glycan structures. In these embodiments, non-sialylated polypeptides are bound to the chromatography matrix and sialylated polypeptides are washed off. The polypeptides obtained from the wash step have a higher amount of sialylation than the initial polypeptides.

Further means for enrichment include methods for separating polypeptides according to their charge. Since sialic acids are negatively charged, sialylated polypeptides can be separated from non-sialylated polypeptides and enriched thereby. Exemplary methods include ion exchange chromatography.

In further embodiments, the method includes an increase of the amount of sialylation of the antibody or fragment, derivative or engraft thereof compared to a reference composition of the antibody or fragment, derivative or engraft thereof. In these embodiments, the antibody or fragment, derivative or engraft thereof which sialylation is to be increased (the reference composition) is produced again using a production method which results in the antibody or fragment, derivative or engraft thereof having a higher amount of sialylation. Suitable means for increasing the amount of sialylation according to these embodiments include, for example, the production of the antibody or fragment, derivative or engraft thereof in a host cell having a higher sialylation activity than the host cell used for production of the reference composition. In certain embodiments, the host cells and methods for production as described herein can be used.

In certain embodiments, the amount of sialylation is increased by at least 10 percent points. In these embodiments, the relative amount of glycans attached to the antibody or fragment, derivative or engraft thereof which comprise at least one sialic acid residue (e.g. at least mono-sialylated on the 3- or 6-arm of the glycan core) is at least 10 percent points higher in the population of the antibody or fragment, derivative or engraft thereof after performing the method for reducing the immunogenicity compared to the population of the antibody or fragment, derivative or engraft thereof prior to performing said method. The amount of sialylation is preferably increased by at least 20 percent points, more preferably by at least 30 percent points, and most preferably by at least 40 percent points.

In certain embodiments, the antibody or fragment, derivative or engraft thereof which immunogenicity is to be reduced has a relative amount of sialylation of 20% or less, preferably 10% or less, more preferably 5% or less, and most preferably 1% or less. The antibody or fragment, derivative or engraft thereof which immunogenicity is to be reduced may for example be produced in a cell which was not engineered for increased expression of an alpha-2,6-sialyltransferase, a beta-1,4-galactosyltransferase and a CMP-sialic acid transporter. In particular, the cell was not engineered for increased expression of any one of an alpha-2,6-sialyltransferase, a beta-1,4-galactosyltransferase and a CMP-sialic acid transporter. In particular, the antibody or fragment, derivative or engraft thereof which immunogenicity is to be reduced is produced in a CHO cell.

The antibody or fragment, derivative or engraft thereof having a reduced immunogenicity in particular is less likely to cause an immune reaction directed against it when administered to a patient. In certain embodiments, the recognition and uptake of the antibody or fragment, derivative or engraft thereof having a reduced immunogenicity by dendritic cells is decreased. In further embodiments, their ability to induce a T cell response against the antibody or fragment, derivative or engraft thereof is decreased. In specific embodiments, the antibody or fragment, derivative or engraft thereof has a reduced ability to cause anti-drug antibodies when administered to a patient.

### SPECIFIC EMBODIMENTS

In the following, specific embodiments of the present invention are described. These embodiments can be combined with the further embodiments, features and examples described herein.

Embodiment 1. A mammalian cell comprising
(i) an exogenous nucleic acid encoding an alpha-2,6-sialyltransferase;
(ii) an exogenous nucleic acid encoding a beta-1,4-galactosyltransferase; and
(iii) an exogenous nucleic acid encoding a CMP-sialic acid transporter.

Embodiment 2. The mammalian cell according to embodiment 1, comprising
(i) a first exogenous expression cassette comprising a first promoter operatively linked to a coding sequence for an alpha-2,6-sialyltransferase;
(ii) a second exogenous expression cassette comprising a second promoter operatively linked to a coding sequence for a beta-1,4-galactosyltransferase; and
(iii) a third exogenous expression cassette comprising a third promoter operatively linked to a coding sequence for a CMP-sialic acid transporter.

Embodiment 3. The mammalian cell according to embodiment 1, comprising
(i) a first exogenous expression cassette comprising a first promoter operatively linked to a coding sequence for an alpha-2,6-sialyltransferase; and
(ii) a second exogenous expression cassette comprising a second promoter operatively linked to a coding sequence for a beta-1,4-galactosyltransferase and to a coding sequence for a CMP-sialic acid transporter.

Embodiment 4. The mammalian cell according to embodiment 3, wherein the second exogenous expression cassette comprises an internal ribosome entry site (IRES) between the coding sequence for a beta-1,4-galactosyltransferase and the coding sequence for a CMP-sialic acid transporter.

Embodiment 5. The mammalian cell according to embodiment 3, wherein the second exogenous expression cassette comprises a coding sequence for a 2A element between the coding sequence for a beta-1,4-galactosyltransferase and the coding sequence for a CMP-sialic acid transporter.

Embodiment 6. The mammalian cell according to any one of embodiments 2 to 5, wherein the first promoter is a strong promoter.

Embodiment 7. The mammalian cell according to any one of embodiments 2 to 6, wherein the first promoter effects expression of the coding sequence for an alpha-2,6-sialyltransferase which results in an amount of transcripts of the coding sequence which is at least as high as the amount of transcripts of a highly expressing housekeeping gene of the mammalian cell, such as EEF1A1 (eukaryotic translation elongation factor 1 alpha 1 gene), ACTB (beta actin gene) and PPIA (peptidylprolyl isomerase A gene).

Embodiment 8. The mammalian cell according to any one of embodiments 2 to 6, wherein the first promoter effects expression of the coding sequence for an alpha-2,6-sialyltransferase which results in an amount of transcripts of the coding sequence which is at least 1.5 times higher than the amount of transcripts of a highly expressing housekeeping gene of the mammalian cell, such as EEF1A1 (eukaryotic translation elongation factor 1 alpha 1 gene), ACTB (beta actin gene) and PPIA (peptidylprolyl isomerase A gene).

Embodiment 9. The mammalian cell according to any one of embodiments 2 to 6, wherein the first promoter effects expression of the coding sequence for an alpha-2,6-sialyltransferase which results in an amount of transcripts of the coding sequence which is at least 2 times higher than the amount of transcripts of a highly expressing housekeeping gene of the mammalian cell, such as EEF1A1 (eukaryotic translation elongation factor 1 alpha 1 gene), ACTB (beta actin gene) and PPIA (peptidylprolyl isomerase A gene).

Embodiment 10. The mammalian cell according to any one of embodiments 2 to 9, wherein the first promoter effects a higher expression than the second promoter and/or the third promoter, if present.

Embodiment 11. The mammalian cell according to embodiment 10, wherein the expression effected by the first promoter is at least 3 times higher than the expression effected by the second promoter and/or the third promoter, if present.

Embodiment 12. The mammalian cell according to any one of embodiments 2 to 11, wherein the first promoter is selected from the group consisting of cytomegalovirus (CMV) promoter, simian virus 40 (SV40) promoter, ubiquitin C (UBC) promoter, elongation factor 1 alpha (EF1A) promoter, phosphoglycerate kinase (PGK) promoter, Rous sarcoma virus (RSV) promoter, BROAD3 promoter, murine rosa 26 promoter, pCEFL promoter and β-actin promoter optionally coupled with CMV early enhancer (CAGG).

Embodiment 13. The mammalian cell according to embodiment 12, wherein the first promoter is a cytomegalovirus (CMV) promoter.

Embodiment 14. The mammalian cell according to any one of embodiments 2 to 13, wherein the second and/or the third promoter is selected from the group consisting of SV40 promoter, CMV promoter, UBC promoter, EF1A promoter, PGK promoter and CAGG promoter.

Embodiment 15. The mammalian cell according to embodiment 14, wherein the second promoter and the third promoter, if present, are a simian virus 40 (SV40) promoter.

Embodiment 16. The mammalian cell according to any one of embodiments 1 to 15, wherein the alpha-2,6-sialyltransferase is beta-galactoside alpha-2,6-sialyltransferase 1 (ST6GAL1), in particular derived from Cricetulus griseus or human.

Embodiment 17. The mammalian cell according to any one of embodiments 1 to 16, wherein the beta-1,4-galactosyltransferase is beta-1,4-galactosyltransferase 1 (B4GALT1), in particular derived from Cricetulus griseus or human.

Embodiment 18. The mammalian cell according to any one of embodiments 1 to 17, wherein the CMP-sialic acid transporter is CMP-sialic acid transporter (SLC35A1), in particular derived from Cricetulus griseus or human.

Embodiment 19. The mammalian cell according to any one of embodiments 1 to 18, wherein the alpha-2,6-sialyltransferase is beta-galactoside alpha-2,6-sialyltransferase 1 (ST6GAL1) of Cricetulus griseus, t beta-1,4-galactosyltransferase is beta-1,4-galactosyltransferase 1 (B4GALT1) of Cricetulus griseus, and the CMP-sialic acid transporter is CMP-sialic acid transporter (SLC35A1) of Cricetulus griseus.

Embodiment 20. The mammalian cell according to embodiment 19, wherein the mammalian cell is a CHO cell.

Embodiment 21. The mammalian cell according to any one of embodiments 1 to 18, wherein the alpha-2,6-sialyltransferase is human beta-galactoside alpha-2,6-sialyltransferase 1 (ST6GAL1), the beta-1,4-galactosyltransferase is human beta-1,4-galactosyltransferase 1 (B4GALT1), and the CMP-sialic acid transporter is human CMP-sialic acid transporter (SLC35A1).

Embodiment 22. The mammalian cell according to embodiment 21, wherein the mammalian cell is a CHO cell.

Embodiment 23. The mammalian cell according to embodiment 21, wherein the mammalian cell is a human cell.

Embodiment 24. The mammalian cell according to any one of embodiments 2 to 23, wherein each expression cassette further comprises a polyadenylation signal (pA).

Embodiment 25. The mammalian cell according to any one of embodiments 1 to 24, wherein the mammalian cell was obtained by transformation with a vector or a combination of two or three vectors comprising the exogenous nucleic acids.

Embodiment 26. The mammalian cell according to embodiment 25, wherein the mammalian cell was obtained by transformation with a vector comprising the first, second and optionally third expression cassette.

Embodiment 27. The mammalian cell according to embodiment 25 or 26, wherein each vector further comprises at least one selectable marker gene.

Embodiment 28 The mammalian cell according to embodiment 27, wherein the selectable marker gene is an antibiotic resistance gene conferring resistance to puromycin, G418, hygromycin, neomycin, zeocin, ouabain, blasticidin, methotrexate (MTX), or methionine sulphoximine (MSX).

Embodiment 29 The mammalian cell according to embodiment 27, wherein the selectable marker gene is a folate receptor gene such as the folate receptor alpha gene, or genes encoding fluorescent proteins such as GFP and RFP.

Embodiment 30. The mammalian cell according to embodiment 27, wherein the selectable marker gene is puromycin-N-acetyltransferase gene (pac).

Embodiment 31. The mammalian cell according to any one of embodiments 27 to 30, wherein one selectable marker gene is positioned on the vector downstream of the expression cassettes, and a second selectable marker, if present, is positioned on the vector upstream of the expression cassettes.

Embodiment 32. The mammalian cell according to embodiment 1, wherein the mammalian cell is a CHO cell stably transfected with a vector comprising
(i) a first exogenous expression cassette comprising a cytomegalovirus (CMV) promoter operatively linked to a coding sequence for beta-galactoside alpha-2,6-sialyltransferase 1 (ST6GAL1) of Cricetulus griseus;
(ii) a second exogenous expression cassette comprising a simian vacuolating virus 40 (SV40) promoter operatively linked to a coding sequence for beta-1,4-galactosyltransferase 1 (B4GALT1) of Cricetulus griseus; and
(iii) a third exogenous expression cassette comprising a simian vacuolating virus 40 (SV40) promoter operatively linked to a coding sequence for CMP-sialic acid transporter (SLC35A1) of Cricetulus griseus.

Embodiment 33. The mammalian cell according to embodiment 32, wherein the vector further comprises the puromycin-N-acetyltransferase gene (pac) as selectable marker gene.

Embodiment 34. The mammalian cell according to embodiment 33, wherein the selectable marker gene is positioned on the vector downstream of the expression cassettes.

Embodiment 35. A mammalian cell, wherein endogenous genes of the mammalian cell encoding an alpha-2,6-sialyltransferase, a beta-1,4-galactosyltransferase and a CMP-sialic acid transporter are engineered for increased expression.

Embodiment 36. The mammalian cell according to embodiment 35, wherein the expression of said endogenous genes is higher compared to the same cell not engineered for increased expression.

Embodiment 37. The mammalian cell according to embodiment 35 or 36, wherein the expression of endogenous nucleic acids encoding an alpha-2,6-sialyltransferase, a beta-1,4-galactosyltransferase and a CMP-sialic acid transporter is upregulated or activated to increase expression.

Embodiment 38. The mammalian cell according to any one of embodiments 35 to 37, wherein increase of expression is obtained by ZFN-activators, TALEN-activators, CRISPR-activators, or chromatin modulation entities.

Embodiment 39. The mammalian cell according to any one of embodiments 35 to 37, wherein increase of expression is obtained by insertion of one or more promoter and/or enhancer elements into genes expressing an alpha-2,6-sialyltransferase, a beta-1,4-galactosyltransferase and a CMP-sialic acid transporter.

Embodiment 40. The mammalian cell according to embodiment 39, comprising
(i) a first exogenous promoter operatively linked to an endogenous coding sequence for an alpha-2,6-sialyltransferase;
(ii) a second exogenous promoter operatively linked to an endogenous coding sequence for a beta-1,4-galactosyltransferase;
(i) a third exogenous promoter operatively linked to an endogenous coding sequence for a CMP-sialic acid transporter.

Embodiment 41. The mammalian cell according to embodiment 40, wherein the first promoter is a strong promoter.

Embodiment 42. The mammalian cell according to embodiment 40 or 41, wherein the first promoter effects expression of the coding sequence for an alpha-2,6-sialyltransferase which results in an amount of transcripts of the coding sequence which is at least as high as the amount of transcripts of a highly expressing housekeeping gene of the mammalian cell, such as EEF1A1 (eukaryotic translation elongation factor 1 alpha 1 gene), ACTB (beta actin gene) and PPIA (peptidylprolyl isomerase A gene).

Embodiment 43. The mammalian cell according to embodiment 40 or 41, wherein the first promoter effects expression of the coding sequence for an alpha-2,6-sialyltransferase which results in an amount of transcripts of the coding sequence which is at least 1.5 times higher than the amount of transcripts of a highly expressing housekeeping gene of the mammalian cell, such as EEF1A1 (eukaryotic translation elongation factor 1 alpha 1 gene), ACTB (beta actin gene) and PPIA (peptidylprolyl isomerase A gene).

Embodiment 44. The mammalian cell according to embodiment 40 or 41, wherein the first promoter effects expression of the coding sequence for an alpha-2,6-sialyltransferase which results in an amount of transcripts of the coding sequence which is at least 2 times higher than the amount of transcripts of a highly expressing housekeeping gene of the mammalian cell, such as EEF1A1 (eukaryotic translation elongation factor 1 alpha 1 gene), ACTB (beta actin gene) and PPIA (peptidylprolyl isomerase A gene).

Embodiment 45. The mammalian cell according to any one of embodiments 40 to 44, wherein the first promoter effects a higher expression than the second promoter and/or the third promoter.

Embodiment 46. The mammalian cell according to embodiment 45, wherein the expression effected by the first promoter is at least 3 times higher than the expression effected by the second promoter and/or the third promoter.

Embodiment 47. The mammalian cell according to any one of embodiments 40 to 46, wherein the first promoter is selected from the group consisting of cytomegalovirus (CMV) promoter, simian virus 40 (SV40) promoter, ubiquitin C (UBC) promoter, elongation factor 1 alpha (EF1A) promoter, phosphoglycerate kinase (PGK) promoter, Rous sarcoma virus (RSV) promoter, BROAD3 promoter, murine rosa 26 promoter, pCEFL promoter and β-actin promoter optionally coupled with CMV early enhancer (CAGG).

Embodiment 48. The mammalian cell according to embodiment 47, wherein the first promoter is a cytomegalovirus (CMV) promoter.

Embodiment 49. The mammalian cell according to any one of embodiments 40 to 48, wherein the second and/or the third promoter is selected from the group consisting of SV40 promoter, CMV promoter, UBC promoter, EF1A promoter, PGK promoter and CAGG promoter.

Embodiment 50. The mammalian cell according to embodiment 49, wherein the second promoter and the third promoter are a simian virus 40 (SV40) promoter.

Embodiment 51. The mammalian cell according to any one of embodiments 1 to 50, wherein the mammalian cell is a CHO cell.

Embodiment 52. The mammalian cell according to any one of embodiments 1 to 50, wherein the mammalian cell is a human cell.

Embodiment 53. The mammalian cell according to any one of embodiments 1 to 52, further comprising an exogenous expression cassette for recombinant expression of a glycosylated polypeptide.

Embodiment 54. The mammalian cell according to embodiment 53, wherein the glycosylated polypeptide is selected from the group consisting of hormones, cytokines, enzymes, antibodies, fusion proteins, vaccines, coagulation proteins, toxins and growth factors.

Embodiment 55. The mammalian cell according to embodiment 53, wherein the glycosylated polypeptide is selected from the group consisting of antibodies and fragments, derivatives or engrafts thereof, in particular proteins comprising an antibody Fc region, whole antibodies, and Fc multimers comprising two or more antibody Fc regions.

Embodiment 56. The mammalian cell according to embodiment 53, wherein the glycosylated polypeptide is an antibody or a protein comprising an antibody Fc region, especially an Fc multimer comprising two or more antibody Fc regions.

Embodiment 57. The mammalian cell according to any one of embodiments 53 to 56, wherein the glycosylated polypeptide is a therapeutic polypeptide or diagnostic polypeptide.

Embodiment 58. A method for producing a glycosylated polypeptide, comprising the steps of
(a) providing a mammalian cell according to any one of embodiments 53 to 57;
(b) cultivating the mammalian cell in a cell culture under conditions which allow for the expression of said glycosylated polypeptide;
(c) obtaining said glycosylated polypeptide from the cell culture; and
(d) optionally processing the glycosylated polypeptide.

Embodiment 59. The method according to embodiment 58, further comprising between steps (a) and (b) the steps of
(a1) inoculating a cell culture medium with the mammalian cell to provide a cell culture, and
(a2) cultivating the mammalian cell in the cell culture under conditions which allow for increasing the number of cells in the cell culture.

Embodiment 60. The method according to embodiment 58 or 59, wherein the temperature is not reduced by more than 2° C. during cultivation of the mammalian cell.

Embodiment 61. The method according to embodiment 58 or 59, wherein the temperature is not altered by more than 2° C. during cultivation of the mammalian cell.

Embodiment 62. The method according to embodiment 58 or 59, wherein the temperature is not reduced by more than 1.5° C. during cultivation of the mammalian cell.

Embodiment 63. The method according to embodiment 58 or 59, wherein the temperature is not altered by more than 1.5° C. during cultivation of the mammalian cell.

Embodiment 64. The method according to any one of embodiments 58 to 63, wherein the culture conditions during cultivation of the mammalian cell do not include a temperature shift.

Embodiment 65. The method according to any one of embodiments 58 to 63, wherein the culture conditions during cultivation of the mammalian cell do not include a temperature shift of more than 2° C.

Embodiment 66. The method according to any one of embodiments 58 to 63, wherein the culture conditions during cultivation of the mammalian cell do not include a temperature shift of more than 1.5° C.

Embodiment 67. The method according to any one of embodiments 58 to 63, wherein the culture conditions during cultivation of the mammalian cell do not include a temperature shift of more than 1° C.

Embodiments 68. The method according to any one of embodiments 58 to 63, wherein the temperature set-point of the cell culture is not changed during cultivation of the mammalian cell.

Embodiment 69. The method according to any one of embodiments 58 to 68, wherein the temperature is kept at 35° C. or more during cultivation of the mammalian cell.

Embodiment 70. The method according to any one of embodiments 58 to 68, wherein the temperature is kept within the range of 34 to 39° C. during cultivation of the mammalian cell.

Embodiment 71. The method according to any one of embodiments 58 to 68, wherein the temperature is kept within the range of 35 to 38° C. during cultivation of the mammalian cell.

Embodiment 72. The method according to any one of embodiments 58 to 71, wherein the step of obtaining the glycosylated polypeptide includes isolating the glycosylated polypeptide from the cell culture.

Embodiment 73. The method according to any one of embodiments 58 to 72, wherein the glycosylated polypeptide is secreted by the mammalian cell and the glycosylated polypeptide is isolated from the cell culture medium.

Embodiment 74. The method according to any one of embodiments 58 to 73, wherein the method comprises step (d) of processing the glycosylated polypeptide.

Embodiment 75. The method according to embodiment 74, wherein processing the glycosylated polypeptide comprises further purification, modification and/or formulation steps.

Embodiment 76. The method according to any one of embodiments 58 to 75, wherein step (d) comprises providing a pharmaceutical formulation comprising the glycosylated polypeptide.

Embodiment 77. The method according to any one of embodiments 58 to 76, wherein the glycosylated polypeptide is an antibody or a fragment, derivative or engraft thereof.

Embodiment 78. A method for producing a glycosylated polypeptide with reduced immunogenicity, comprising the steps of
(a) providing a mammalian cell according to the first aspect of the present invention which further comprises an expression cassette for recombinant expression of a glycosylated polypeptide;
(b) cultivating the mammalian cell in a cell culture under conditions which allow for the expression of said glycosylated polypeptide;
(c) obtaining said glycosylated polypeptide from the cell culture; and
(d) optionally processing the glycosylated polypeptide;
wherein the glycosylated polypeptide is an antibody or a fragment, derivative or engraft thereof.

Embodiment 79. The method according to embodiment 78, having any one or more of the features as defined in embodiments 59 to 77.

Embodiment 80. The method according to embodiment 78 or 79, wherein the glycosylated polypeptide has a reduced immunogenicity compared to a reference glycosylated polypeptide which has the same amino acid sequence as the glycosylated polypeptide with reduced immunogenicity, but a lower amount of sialylation.

Embodiment 81. The method according to embodiment 80, wherein the amount of sialylation of the reference glycosylated polypeptide is at least 10 percent points lower, preferably at least 20 percent points lower, more preferably at least 30 percent points lower, and most preferably at least 40 percent points lower.

Embodiment 82. The method according to embodiment 80 or 81, wherein the reference glycosylated polypeptide has an amount of sialylation of 5% or less, preferably of 1% or less.

Embodiment 83. A vector nucleic acid or a combination of at least two vector nucleic acids, comprising
(i) a coding sequence for an alpha-2,6-sialyltransferase;
(ii) a coding sequence for a beta-1,4-galactosyltransferase; and
(iii) a coding sequence for a CMP-sialic acid transporter.

Embodiment 84. The vector nucleic acid or combination of at least two vector nucleic acids according to embodiment 83, wherein the coding sequences are part of one or more expression cassettes, each comprising a promoter which is operatively linked to the coding sequence(s), and wherein the expression cassettes are for expression in a mammalian host cell.

Embodiment 85. The vector nucleic acid or combination of at least two vector nucleic acids according to embodiment 83 or 84, wherein each coding sequence is part of a separate expression cassette or two or three of the coding sequences are part of the same expression cassette.

Embodiment 86. The vector nucleic acid or combination of at least two vector nucleic acids according to any one of embodiments 83 to 85, comprising
(i) a first expression cassette comprising a first promoter operatively linked to the coding sequence for an alpha-2,6-sialyltransferase;
(ii) a second expression cassette comprising a second promoter operatively linked to the coding sequence for a beta-1,4-galactosyltransferase; and
(iii) a third expression cassette comprising a third promoter operatively linked to the coding sequence for a CMP-sialic acid transporter.

Embodiment 87. The vector nucleic acid or combination of at least two vector nucleic acids according to any one of embodiments 83 to 85, comprising
(i) a first expression cassette comprising a first promoter operatively linked to the coding sequence for an alpha-2,6-sialyltransferase; and
(ii) a second expression cassette comprising a second promoter operatively linked to the coding sequence for a beta-1,4-galactosyltransferase and to the coding sequence for a CMP-sialic acid transporter.

Embodiment 88. The vector nucleic acid or combination of at least two vector nucleic acids according to embodiment 87, wherein the second exogenous expression cassette comprises an internal ribosome entry site (IRES) or a coding sequence for a 2A element between the coding sequence for a beta-1,4-galactosyltransferase and the coding sequence for a CMP-sialic acid transporter.

Embodiment 89. The vector nucleic acid or combination of at least two vector nucleic acids according to any one of embodiments 86 to 88, wherein the first promoter effects a higher expression than the second promoter and/or the third promoter, if present.

Embodiment 90. The vector nucleic acid or combination of at least two vector nucleic acids according to embodiment 89, wherein the expression effected by the first promoter is at least 3 times higher than the expression effected by the second promoter and/or the third promoter, if present.

Embodiment 91. The vector nucleic acid or combination of at least two vector nucleic acids according to any one of embodiments 86 to 90, wherein the first promoter is a cytomegalovirus (CMV) promoter.

Embodiment 92. The vector nucleic acid or combination of at least two vector nucleic acids according to any one of embodiments 86 to 91, wherein the second and/or the third promoter is a simian vacuolating virus 40 (SV40) promoter.

Embodiment 93. The vector nucleic acid or combination of at least two vector nucleic acids according to any one of embodiments 83 to 92, wherein the alpha-2,6-sialyltransferase is beta-galactoside alpha-2,6-sialyltransferase 1 (ST6GAL1), in particular derived from Cricetulus griseus or human.

Embodiment 94. The vector nucleic acid or combination of at least two vector nucleic acids according to any one of embodiments 83 to 93, wherein the beta-1,4-galactosyltransferase is beta-1,4-galactosyltransferase 1 (B4GALT1), in particular derived from Cricetulus griseus or human.

Embodiment 95. The vector nucleic acid or combination of at least two vector nucleic acids according to any one of embodiments 83 to 94, wherein the CMP-sialic acid transporter is CMP-sialic acid transporter (SLC35A1), in particular derived from Cricetulus griseus or human.

Embodiment 96. The vector nucleic acid or combination of at least two vector nucleic acids according to embodiment 83, being a vector for stable transfection of a host cell, comprising
(i) a first exogenous expression cassette comprising a cytomegalovirus (CMV) promoter operatively linked to a coding sequence for beta-galactoside alpha-2,6-sialyltransferase 1 (ST6GAL1) of Cricetulus griseus;
(ii) a second exogenous expression cassette comprising a simian vacuolating virus 40 (SV40) promoter operatively linked to a coding sequence for beta-1,4-galactosyltransferase 1 (B4GALT1) of Cricetulus griseus; and
(iii) a third exogenous expression cassette comprising a simian vacuolating virus 40 (SV40) promoter operatively linked to a coding sequence for CMP-sialic acid transporter (SLC35A1) of Cricetulus griseus.

Embodiment 97. The vector nucleic acid or combination of at least two vector nucleic acids according to any one of embodiments 83 to 96, wherein each expression cassette further comprises a polyadenylation signal (pA).

Embodiment 98. The vector nucleic acid or combination of at least two vector nucleic acids according to any one of embodiments 83 to 97, wherein each vector nucleic acid further comprises at least one selectable marker gene.

Embodiment 99. The vector nucleic acid or combination of at least two vector nucleic acids according to embodiment 98, wherein the selectable marker gene is an antibiotic resistance gene such as puromycin-N-acetyltransferase gene (pac).

Embodiment 100. The vector nucleic acid or combination of at least two vector nucleic acids according to embodiment 98 or 99, wherein one selectable marker gene is positioned on the nucleic acid downstream of the expression cassettes, and a second selectable marker, if present, is positioned on the nucleic acid upstream of the expression cassettes.

Embodiment 101. The vector nucleic acid or combination of at least two vector nucleic acids according to any one of embodiments 83 to 100, for stable transfection of a host cell.

Embodiment 102. Use of the vector nucleic acid or combination of at least two vector nucleic acids according to any one of embodiments 83 to 101 for the transfection of a mammalian cell.

Embodiment 103. The use according to embodiment 102, wherein the mammalian cell is a Chinese hamster ovary (CHO) cell.

Embodiment 104. A method for increasing expression of an alpha-2,6-sialyltransferase, a beta-1,4-galactosyltransferase and a CMP-sialic acid transporter in a mammalian cell, comprising the step of transfecting the mammalian cell with the vector nucleic acid or a combination of at least two vector nucleic acids according to any one of embodiments 83 to 101.

Embodiment 105. The method according to embodiment 104, comprising the steps of
(i) providing a mammalian cell;
(ii) transfecting the mammalian cell with the vector nucleic acid or a combination of at least two vector nucleic acids according to any one of embodiments 83 to 101;
(iii) obtaining an engineered mammalian cell with increased expression of an alpha-2,6-sialyltransferase, a beta-1,4-galactosyltransferase and a CMP-sialic acid transporter.

Embodiment 106. The method according to embodiment 105, wherein the engineered mammalian cell is a mammalian cell as defined in any one of embodiments 1 to 34 and 51 to 57.

Embodiment 107. A method for increasing expression of an alpha-2,6-sialyltransferase, a beta-1,4-galactosyltransferase and a CMP-sialic acid transporter in a mammalian cell, comprising the step of engineering endogenous genes of the mammalian cell encoding an alpha-2,6-sialyltransferase, a beta-1,4-galactosyltransferase and a CMP-sialic acid transporter for increased expression.

Embodiment 108. The method according to embodiment 107, comprising the steps of
(i) providing a mammalian cell;
(ii) insertion of one or more promoter and/or enhancer elements into a gene expressing an alpha-2,6-sialyltransferase, into a gene expressing a beta-1,4-galactosyltransferase and into a gene expressing a CMP-sialic acid transporter;
(iii) obtaining an engineered mammalian cell with increased expression of an alpha-2,6-sialyltransferase, a beta-1,4-galactosyltransferase and a CMP-sialic acid transporter.

Embodiment 109. The method according to embodiment 108, wherein the engineered mammalian cell is a mammalian cell as defined in any one of embodiments 35 to 57.

Embodiment 110. A method for reducing the immunogenicity of an antibody or a fragment, derivative or engraft thereof, comprising the step of increasing the amount of sialylation in the glycosylation pattern of the antibody or fragment, derivative or engraft thereof.

Embodiment 111. The method according to embodiment 110, wherein the antibody or fragment, derivative or engraft thereof is a therapeutic antibody or fragment, derivative or engraft thereof.

Embodiment 112. The method according to embodiment 111 for reducing the immunogenicity of a therapeutic antibody.

Embodiment 113. The method according to any one of embodiments 110 to 112, wherein the antibody or fragment, derivative or engraft thereof comprises a CH2 domain with an N-glycosylation site including the asparagine residue at amino acid position 297 of the antibody heavy chain according to the Kabat numbering, and the step of increasing the amount of sialylation includes increasing the amount of sialylation in the N-glycosylation pattern of said CH2 domain.

Embodiment 114. The method according to any one of embodiments 110 to 113, wherein the step of increasing the amount of sialylation includes treating the antibody or fragment, derivative or engraft thereof so that sialic acid residues are attached to the glycans present on the antibody or fragment, derivative or engraft thereof.

Embodiment 115. The method according to embodiment 114, wherein the step of increasing the amount of sialylation includes in vitro treatment of the antibody or fragment, derivative or engraft thereof with a sialyltransferase such as alpha-2,6-sialyltransferase, and a sialic acid donor.

Embodiment 116. The method according to any one of embodiments 110 to 115, wherein the step of increasing the amount of sialylation includes enriching those antibody or fragment, derivative or engraft thereof which carry at least one sialic acid.

Embodiment 117. The method according to embodiment 116, wherein the step of increasing the amount of sialylation includes performing one or more of
(i) affinity chromatography using ligands specifically binding to sialylated glycan structures;
(ii) affinity chromatography using ligands specifically binding to non-sialylated glycan structures; and
(iii) ion exchange chromatography.

Embodiment 118. The method according to any one of embodiments 110 to 117, wherein the step of increasing the amount of sialylation includes producing the antibody or fragment, derivative or engraft thereof using a production method which results in a higher amount of sialylation compared to a reference composition of the antibody or fragment, derivative or engraft thereof.

Embodiment 119. The method according to embodiment 118, wherein the antibody or fragment, derivative or engraft thereof is produced in a host cell having a higher sialylation activity than the host cell used for production of the reference composition.

Embodiment 120. The method according to embodiment 119, wherein the host cell used for production of the antibody or fragment, derivative or engraft thereof with reducing the immunogenicity is a mammalian cell according to any one of embodiments 1 to 53.

Embodiment 121. The method according to embodiment 119 or 120, wherein the antibody or fragment, derivative or engraft thereof with reducing the immunogenicity is produced by a method according to any one of embodiments 58 to 82.

Embodiment 122. The method according to any one of embodiments 110 to 121, wherein the amount of sialylation is increased by at least 10 percent points.

Embodiment 123. The method according to any one of embodiments 110 to 121, wherein the amount of sialylation is increased by at least 20 percent points.

Embodiment 124. The method according to any one of embodiments 110 to 121, wherein the amount of sialylation is increased by at least 30 percent points.

Embodiment 125. The method according to any one of embodiments 110 to 121, wherein the amount of sialylation is increased by at least 40 percent points.

Embodiment 126. The method according to any one of embodiments 110 to 125, wherein the antibody or fragment, derivative or engraft thereof which immunogenicity is to be reduced has a relative amount of sialylation of 20% or less.

Embodiment 127. The method according to any one of embodiments 110 to 125, wherein the antibody or fragment, derivative or engraft thereof which immunogenicity is to be reduced has a relative amount of sialylation of 10% or less.

Embodiment 128. The method according to any one of embodiments 110 to 125, wherein the antibody or fragment, derivative or engraft thereof which immunogenicity is to be reduced has a relative amount of sialylation of 5% or less.

Embodiment 129. The method according to any one of embodiments 110 to 125, wherein the antibody or fragment, derivative or engraft thereof which immunogenicity is to be reduced has a relative amount of sialylation of 2% or less.

Embodiment 130. The method according to any one of embodiments 110 to 129, wherein the antibody or fragment, derivative or engraft thereof having a reduced immunogenicity is less likely to cause an immune reaction directed against it when administered to a patient.

Embodiment 131. The method according to any one of embodiments 110 to 130, wherein the recognition and uptake of the antibody or fragment, derivative or engraft thereof having a reduced immunogenicity by dendritic cells is decreased.

Embodiment 132. The method according to any one of embodiments 110 to 131, wherein the ability of the antibody or fragment, derivative or engraft thereof having a reduced immunogenicity to induce a T cell response against it and/or to induce anti-drug antibodies against it is decreased.

### FIGURES

FIG. 1 shows the experimental design of hypersialylation evaluation. Eight different vectors were transfected in three different CHO clones expressing “one armed antibody” molecule.

FIG. 2 shows the different vector strategies schematically (left; I=ST6Gal-I, II=B4galt1 and III=Slc35a1). The right hand side lists the Fc-glycan profiling results from mass spectrometric analysis of protein-A purified one-armed antibody pools. The first column shows the vector strategy, the second column the performed transfections and how many pools have recovered selection phase, the other columns the relative percentage of the specific glycans and sialoglycans.

FIG. 3 shows the productivity (titer) for the “non glycoengineered” control clone and the same clone stably transfected with the different glycovectors.

FIG. 4 shows schematically the different vector strategies (which were transfected in a CHO cell line; left). On the right hand side the relevant glycoprofile data of the transient transfected three Fc fusion protein (“Fc-trimer”) are highlighted. The first column shows the vector strategy, the second column the number of performed transfections and the other columns the respective percentage values of the specific glycosylation (only 2,6-sialoglycans are shown).

FIG. 5 shows schematically the different vector strategies which were transfected in a different CHO clone compared to FIG. 4 (left). On the right hand side the relevant glycoprofile data of the transient transfected Fc-trimer are highlighted. The first column shows the vector strategy, the second column the performed transfections and the other columns the respective percentage values of the specific glycosylation (only 2,6-linked sialoglycans are shown).

FIG. 6 shows the titer of CHO as well as glycoengineered CHO (geCHO) of the five Fc fusion protein (“Fc-pentamer”) and the variant thereof with a point mutation.

FIG. 7 shows the glycoprofile of the Fc-pentamer (expressed in CHO and geCHO) and the variant thereof with a point mutation (expressed in geCHO). The first column shows the performed transfections and the other columns the respective percentage values of the specific glycosylation (only 2,6-sialoglycans shown).

FIG. 8 shows the experimental design to determine relevant factors for glycosylation.

FIG. 9 shows productivity (titer) for the OAA clone stably transfected with different glycovectors.

FIG. 10 shows sialylation level for the OAA clone stably transfected with different glycovectors (average and standard deviation of three transfections/each).

FIG. 11 shows titer data of Fc-trimer complex expressed in geCHO (clone).

FIG. 12 shows the level of sialylation of Fc-trimer complex expressed in geCHO (clone).

FIG. 13 shows titer data of two Fc-trimer complex expressed in parental CHO and geCHO (clone).

FIG. 14 shows the level of sialylation of Fc-trimer complex expressed in parental CHO and geCHO (clone).

FIG. 15 shows titer data (fedbatch) of different products (2-3 biol. replicates/each).

FIG. 16 shows the level of 2,6- as well as 2,3-sialylation of different products (2-3 biol. replicates/each).

FIG. 17 shows growth (A), viability (B), product concentration (C) and specific productivity qp (D) for the process conditions 1 (squares) and 2 (circles) in a 10 L bench scale bioreactor.

FIG. 18 shows the glycan species of two different process conditions (left: process condition 2, right: process condition 1) over time (days 7, 10, 13, and 14). bG0 is a sum of bG0-GlcNac-F (%), bG0-GlcNac (%), bG0-F (%) and bG0 (%). bG1 is a sum of bG1-GlcNac, bG1-F (%), bG1-F (%), 1.6-bG1 (%), and 1.3-bG1 (%). Mannose species is a sum of M5 (%) and M6 (%). 2.3 Sial(ylation) is a sum of bG1-2.3-S1 (%), bG2-2.3-S1 (%) and bG2-2.3-S2 (%). Overall 2.6 Sial(ylation) is a sum of bG1-2.3-S1 (%), 1.6 bG1-2.6-S1 (%), 1.3 bG1-2.6-S1 (%), bG2-2.3-S1 (%), bG2-2.6-S1 (%), bG2-2.3-S2 (%), bG2-2.3/2.6-S2 (%) and bG2-2.6-S2 (%).

FIG. 19 shows viable cell concentration, viability and product concentration (titer) of the co-cultivation of geCHO parental cells with (squares) and without product (circles) over the cultivation period of 14 days in a 100 mL shake flask in fed-batch mode.

FIG. 20 shows the average of the glycan species over the cultivation period of 14 days in a 100 mL shake flask in fed-batch mode of the spiked product (Fc-trimer).

FIG. 21 shows FPKM values of endogenous expressed St6gal1, B4galt1 and Slc35a1, a list of housekeeping genes as well as the mean and median value of all expressed genes for the parental CHO cell line.

FIG. 22 shows FPKM values of exogenous and endogenous expressed St6gal1, B4galt1 and Slc35a1, a list of housekeeping genes, as well as the mean and median value of all expressed genes for the parental geCHO cell line.

FIG. 23 shows viability during selection of CHO cells transfected with vector p001.

FIG. 24 shows internalization of mAbX1 variants by IDCs measured by indirect FACS method. A: DC-internalization of WT, N297A, mannosylated (HiMan) and hypersialylated (HySi) mAbX1 at 60 min; normalized from Delta 4° C. and 37° ° C. of % mAbX1 on viable IDCs, n=6 human donors; statistical analysis conducted by Kruskal-Wallis chi-squared test; B: Binding of mAbX1 glycovariants to antigen-positive Ramos cells.

FIG. 25 shows binding and internalization of mAbX1 glycovariants detected by confocal microscopy and real-time imaging. A: Quantification of intracellular mAbX1 staining from confocal images after DC uptake of mAbX1 glycovariants at 37° C. calculated with HALOT image analysis software (Akoya Biosciences®), n=3 images per variant. B: Kinetic of internalization of mAbX1 glycovariants by IDCs using the IncuCyte Real Time Imager (median from 25 human donors, median Intensity: fluorescence signal derived from AF647-labelled mAbX1 glycovariants indicating internalization).

FIG. 26 shows T cell response to mAbX1 glycovariants. A: Count of proliferating and CD25+ Th cells determined from PBMCs of 16 human naive donors primed and challenged with of each the mAbX1 glycovariant (10 μg/mL); B: Response index for T cell assay in the same donor set, responding donor: donor with stimulation index above 1.5 (dashed line); C: SI for WT responders; D: Representative dot plots of proliferating CD25+ Th cells with (+) or without (−) challenge (=prime response) from one human donor.

### EXAMPLES

### Example 1: Glycoengineering of CHO Cells

A variety of cell line engineering strategies were evaluated to produce hypersialylated proteins in CHO host cell line. These included the overexpression of one, two or three relevant genes derived from Cricetulus griseus:
1: ST6Gal-I: CHO glycosylation machinery is very similar to that found in human cells, but lack alpha-2,6-sialyltransferase-I activity (ST6Gal-I). This gene is responsible for the addition of sialic acids on galactose residues with an α-2,6 linkage.
2: B4galt1: beta-1,4-galactosyltransferase (B4galt1) is responsible for catalyzing the transfer of galactose to glycol-proteins and therefore for the synthesis of complex-type N-linked oligosaccharides (increase of bG1 and bG2).
3: Overexpression of Slc35a1: Overexpressing CMP-sialic acid transporter (CMPSAT) (a nucleotide sugar transporter) could improve the sialylation process in Chinese hamster ovary cells (CHO) through increasing the transport of CMP-sialic acid into the Golgi, resulting in an increased CMP-sialic acid intra-lumenal pool and increased sialylation of the proteins produced.

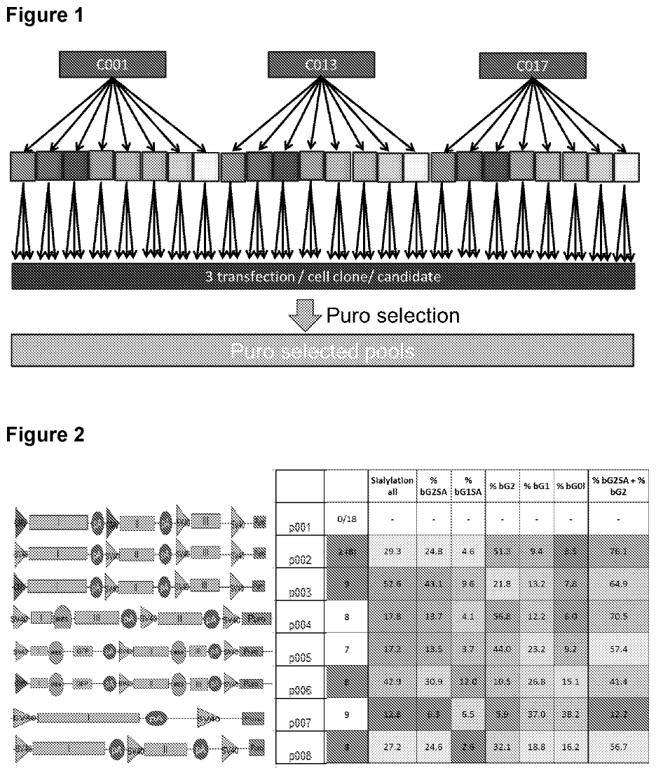

A variety of promoters, IRES elements, GFP as selectable marker and combinations of these three genes were evaluated to increase 2,6-sialylation. Overall 9 vectors were generated and stably transfected in three CHO clones expressing a “one-armed antibody” format (experimental setup see FIG. 1).

For all vector strategies puromycin was used as selectable marker. Fc-glycan profiling was performed by mass spectrometric analysis of protein-A purified one-armed antibody pools. The glycoanalysis data of this evaluation experiment are summarized in the FIG. 2. The MS method is semi-quantitative and was used to rapidly screen the pool samples. For the following experiments, the more accurate released glycan method using 2-AB labeling and HILIC-FLD chromatography was used which can differentiate between 2,6- and 2,3-linked sialoglycans.

Surprisingly the highest sialylation could be achieved applying strategies using vectors “p003” and “p006”; both having the strong CMV promoter upstream of ST6Gal-I and additionally expression of the genes B4galt1 and Slc35a1 (downstream of medium strong SV40 promoters). The total sialylation measured with mass spectrometry was in average 52.5% and 42.9%, respectively. Applying 2-AB HILIC-FLD method confirmed these data. Furthermore using 2,3- and 2,6-linked sialoglycan reference standards enabled the discrimination of 2,3-linked and 2,6-linked sialic acid. 2,6-linked sialoglycans were the dominant forms (less than 5% is 2,3-linked sialic acid, corresponding to the overall sialylation level of the parental CHO cells).

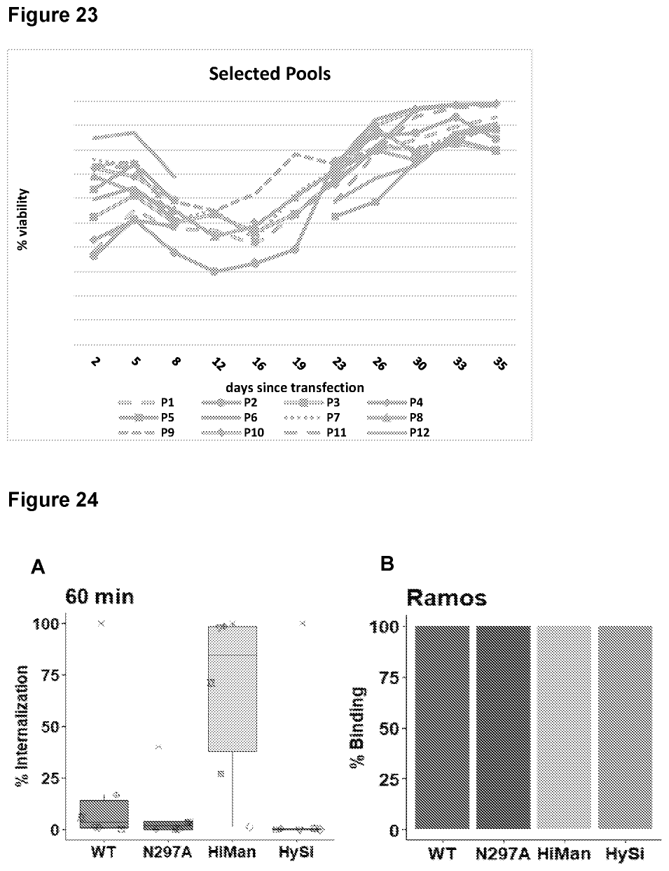

In a first approach, transfecting vector “p001” resulted in no surviving pools, although twice the numbers of transfections were performed compared to the other strategies. This is the only strategy having the strong CMV promoter upstream of B4galt1. Microarray transcriptomics data (of the CHO cell line used for this experiment) highlighted that ST6Gal-I is not expressed, B4galt1 is very low and Slc35a1 medium expressed. Therefore we would have expected that a “high overexpression” of B4galt1 (using CMV promoter) would be beneficial for high sialylation level and it was unexpected that “high overexpression” of B4galt1 resulted in non-surviving pools, whereas “medium-high overexpression” (using SV40 promoter) did not show a negative impact. However, in a second approach the cells survived the transfection/selection phase with vector p001. Overall, the selection crisis was longer than usual, but after 35 days all pools had viability above 80% (see FIG. 23). Therefore, we were able to generate stable transfected parental cells with this plasmid.

The relevance of overexpressing the gene B4galt1 is evident considering the glycosylation data of vector strategy “p007”. This was the only strategy without overexpressing B4galt1. Missing overexpression of B4galt1 resulted in higher amounts of bG0 (38%) compared to the other strategies (4%-16%). The galactosyl group is the substrate for the terminal sialylation; therefore strategy “p007” resulted also in the lowest sialylation compared to the other strategies.

### Example 2: Productivity of the Glycoengineered Cells

Also a relevant topic is the productivity of the glycoengineered cell lines. Therefore, it was determined if the overexpression of the three genes have any impact on productivity compared to the non glycoengineered control.

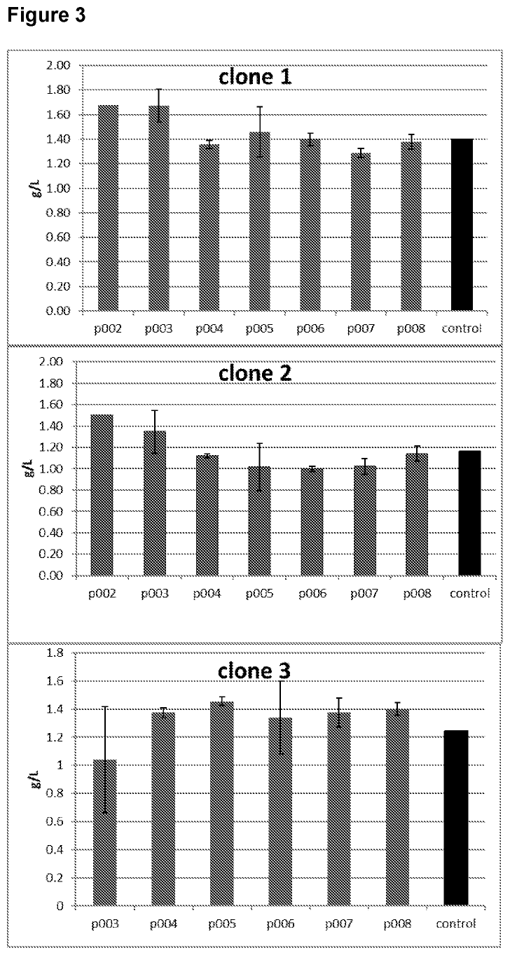

As shown in FIG. 3 no impact on productivity was detected after stable transfection of one armed antibody clone (control) with the different “glycoengineering vectors”. Expression with three different one armed antibody expressing clones was performed and in none of the clones a positive or negative effect on productivity could be measured compared to the non-glycoengineered cell line.

### Example 3: Stable Transfection of CHO Cell Lines

In the next step, the four best vector strategies were stably transfected into a parental CHO cell line clone (strategies p002, p003, p006 and p008). Three to five transfections per strategy were performed and stable pools were generated. To evaluate these pools in respect to their capability of sialylation, transient transfections of a fusion protein comprising three Fc-fragments (“Fc-trimer”) was performed. The glycoprofiles were determined via 2-AB HILIC-FLD method.

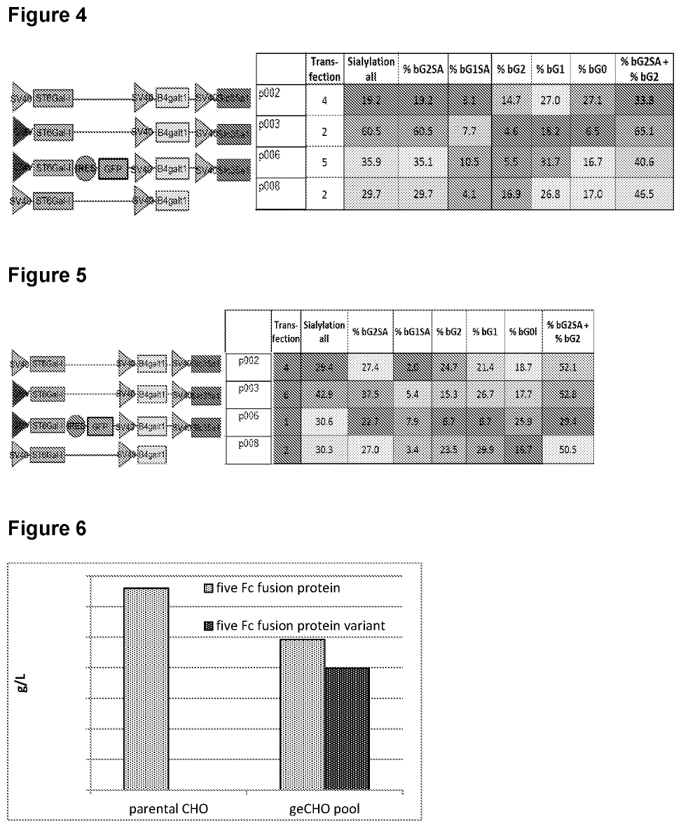

For all vector strategies puromycin was used as selectable marker. The results are shown in FIG. 4. The data were well correlating to the first evaluation study with the one-armed antibody. The vector strategy with p003 (strong CMV promoter upstream of ST6Gal-I and additionally expression of the genes B4galt1 and Slc35a1) resulted in the highest sialylation level (2,6 sialylation levels are shown in FIG. 4). The majority of the glycopattern is bG2SA (biantennary complex-type glycans with 2 galactoses and at least 1 sialic acid): 32% are bG2S2 (biantennary complex-type glycans with 2 galactoses and 2 sialic acids) and 29% are bG2S1 (biantennary complex-type glycans with 2 galactoses and 1 sialic acid). Although all four strategies were using the same SV40 promoter upstream of B4galt1, strategy p003 resulted surprisingly in significant lower amounts of bG0 and bG1 (biantennary complex-type glycans with 0 or 1 galactose, respectively) compared to the other strategies.

Similar data were achieved with transfection of the same four vectors in a different parental CHO clone, which is less suitable for expression of therapeutic proteins (lower titers) and also showed overall lower level of sialylation (see FIG. 5). Nevertheless strategy p003 showed also here the highest level of sialylation and the majority of the glycopattern is bG2SA (16% were bG2S2 and 20% bG2S1).

Another set of experiments was done with different sample proteins. Both stable pools transfected with p003 (see FIG. 4) were mixed and subsequently stably transfected with vectors encoding a fusion protein comprising five Fc-fragments (“Fc-pentamer”). The same was done with a variant of the Fc-pentamer with a point mutation in the Fc part (the single amino acid mutation in the Fc part is known to enhanced galactosylation and sialylation). Additionally this Fc-pentamer was also transfected in a parental CHO cell clone. After one selection step, productivity was determined as well as glycan analytics was performed.

Very high productivity could be measured for both cell lines and for all constructs. The titers of the Fc-pentamer as well as the variant with a point mutation were comparable in geCHO (see FIG. 6).

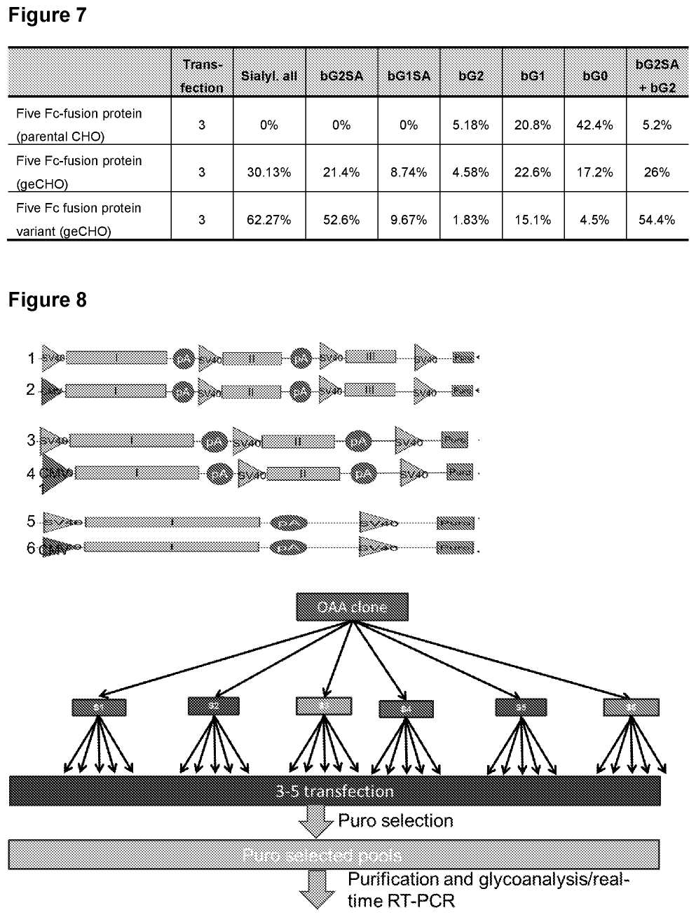

The glycoanalysis of the Fc-pentamer showed that no 2,6-sialylation could be detected in the parental CHO cell clone (as expected) (FIG. 7). In contrast the Fc-pentamer as well as the variant expressed in geCHO are 2,6-sialylated. The overall 2,6-linked sialylation of the Fc-pentamer is lower (2,6-linked total sialylation approx. 30%) compared to the transient transfection of the Fc-trimer in the same pools (approx. 60%). This might be due to the more complex molecule (pentamer instead of trimer) or the missing selection pressure during the selection phase for the Fc-pentamer (only the selection agent for the Fc-pentamer was applied, but not the selection agent for the p003 construct (puromycin)). Nevertheless, the point mutation variant showed a high sialylation of up to more than 60%. Whereas in all the examples above the distribution of bG2S2 and bG2S1 were roughly the same, the Fc-pentamer had less than 1% bG2S2 and 21% bG2S1. In contrast the variant had 40% bG2S2 and 13% bG2S1.

So far it could clearly be shown that a vector strategy with a strong promoter, as e.g. the CMV promoter, upstream of ST6Gal-I and additionally the overexpression of the genes B4galt1 and Slc35a1 with a medium strong promoter, as e.g. SV40 promoter, without any additional elements, as e.g. IRES, resulted in the highest sialylation. The second highest sialylation were detect with a similar vector approach, but having downstream of ST6GAL-1 an IRES element followed by a GFP cassette. The direct comparison of CMV promoter upstream of ST6Gal-I versus SV40 promoter upstream of ST6Gal-I showed, that the CMV promoter driven ST6Gal-I expression results in a significant higher sialylation.

Additionally the overexpression of the B4galt1 is required for increased sialylation. Sialic acids are attached to terminal galactose residues of N-glycan structures. A higher B4galt1 activity increases the amount of such galactose residues and hence, provides more attachment sites for sialic acids. The increase sialylation activity provided by overexpression of the alpha-2,6-sialyltransferase ST6GAL-1 and the CMP-sialic acid transporter Slc35a1 hence is further improved by overexpression of the beta-1,4-galactosyltransferase B4galt1. Applying strategy p007 (only overexpressing ST6GAL-1 without overexpression of B4galt1) resulted in the lowest sialylation levels.

### Example 4: Relevance of the Sialic Acid Transporter

Slc35a1 is required for increasing the transport of CMP-sialic acid into Golgi resulting in an increased CMP-sialic acid intra-lumenal pool. Having the weaker SV40 promoter upstream of ST6Gal-I in p002 and p008, we speculated that the intra-lumenal CMP-sialic acid is not yet a limiting factor and therefore it does not make a difference if this gene is expressed or not. But having the very strong CMV promoter upstream of ST6Gal-I, intra-lumenal CMP-sialic acid might become a limiting factor and overexpression of Slc35a1 might become beneficial. To evaluate this factor the comparison shown in FIG. 8 was performed.

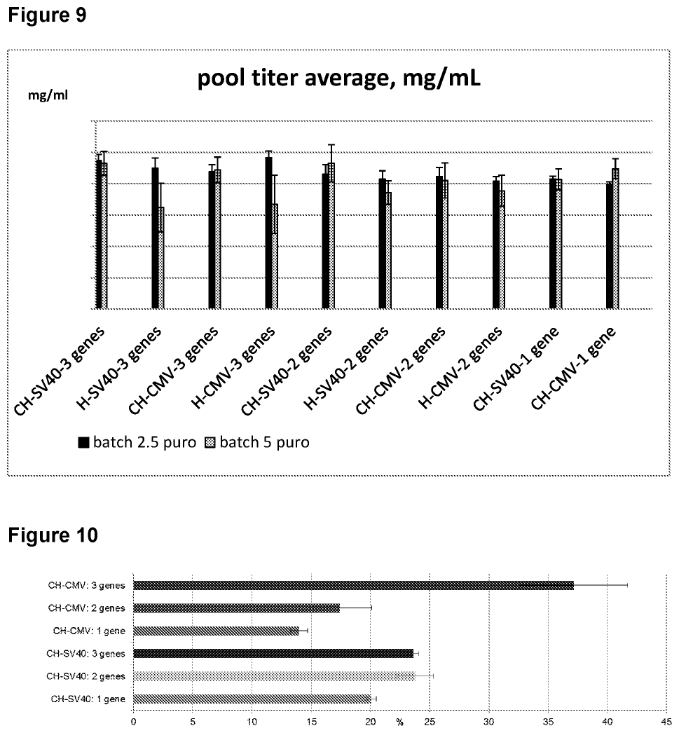

As a result, no difference in productivity (titer) could be detected for any of the glycostrategies (see FIG. 9). FIG. 10 shows the sialylation level for the different strategies (overexpression of one, two or three “glycoengineering genes” (incl. overexpression of ST6Gal-I using SV40 or CMV promoter)). Overexpression of ST6Gal-I only using the SV40 promoter resulted in the lowest sialylation level (see FIG. 10). Additional overexpression of B4galt1 or B4galt1 plus Slc35a1 resulted in elevated sialylation level. This is very likely due to the increase of galactose as substrate for further sialylation (level of bG0 is between 31%-34% if only ST6Gal-I is expressed and bG0 level is reduced to 14%-20% if B4galt1 or B4galt1 plus Slc35a1 are additionally expressed). There is no difference if Slc35a1 is expressed or not indicating that sialic acid is not limited in the Golgi.

Overexpression of ST6Gal-I only or ST6Gal-I plus B4galt1 using the strong CMV promotor upstream of ST6Gal-I did not result in any increased sialylation compared to the same vector strategy using the SV40 promoter. Surprisingly a significant increase of sialylation was detected as soon as Slc35a1 is additionally expressed next to ST6Gal-I (downstream of the CMV promotor) and B4galt1 (see FIG. 10). It seems that the amount of sialic acid within the Golgi is limiting high sialylation as long as Slc35a1 is not overexpressed.

### Example 5: Expression of Different Products in the Glycoengineered CHO Cell Lines

The parental CHO and the geCHO pool (stable transfected with the vector encoding ST6Gal-I (downstream of a CMV promoter), B4galt1 and Slc35a1 as shown in FIG. 5) were evaluated with different projects up to pool level.

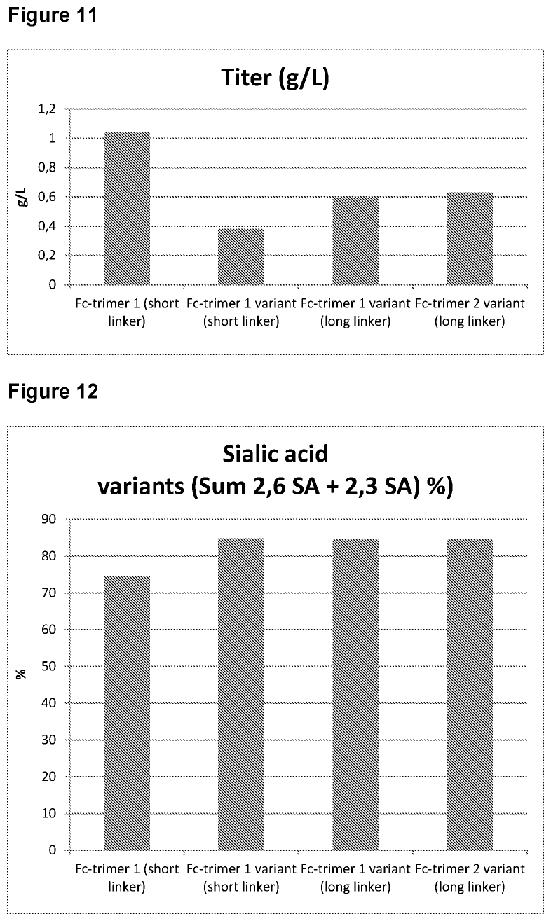

Four Fc-trimer protein constructs (with different Fc-multimerisation domains, linker length and an amino acid exchange for three constructs) were expressed in a geCHO clone. The expression of the construct without the amino acid exchange resulted in the highest titer and the lowest sialylation (almost 2 fold higher titer, but 10% lower level of total sialylation) (FIGS. 11 and 12). The amino acid exchange is known to have an enhanced effector function. Nevertheless, the titer was for all constructs in a good range and the sialylation levels surprisingly high. These data highlight, that the geCHO clone is capable of good productivity for such a complex molecule and high sialylation levels.

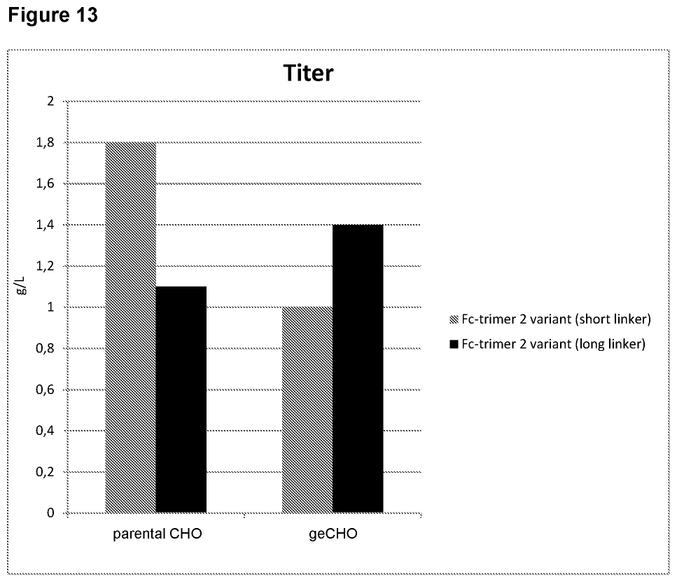

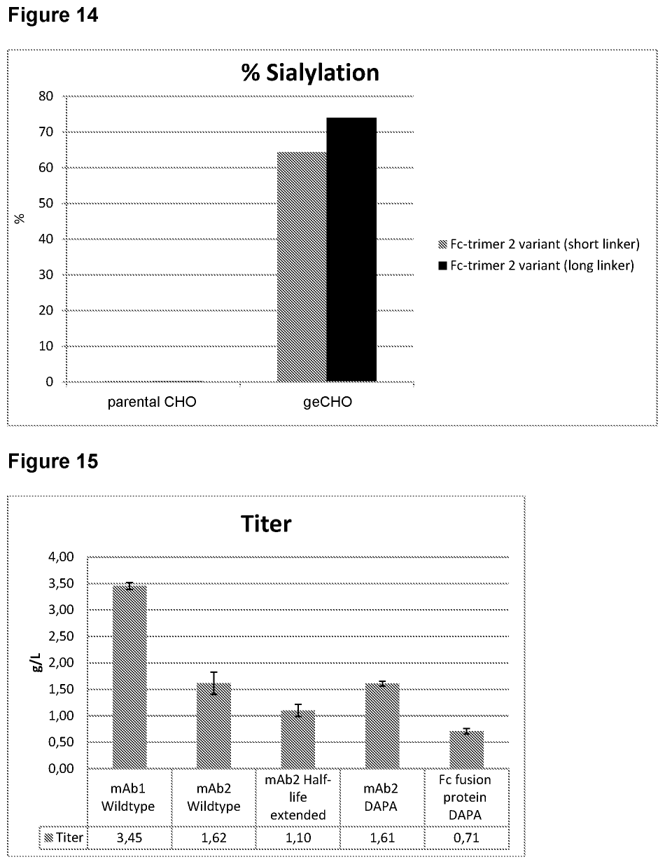

Furthermore, two Fc-trimer protein constructs (only difference is linker length) were expressed in parental CHO and in a geCHO clone. Both constructs have the amino acid exchange. The expression level of CHO and geCHO were comparable (for one Fc-trimer construct CHO showed higher titer, for the other construct geCHO had higher titer) (FIGS. 13 and 14). The overall sialylation levels were for the construct with the short linker around 65% and for the construct with the long linker ca. 75% (if expressed in geCHO clone) (the amount of 2,3-sialylation level was under 5%). In cyno and mouse studies, the higher amount of sialylation showed to be more efficacious and resulted in longer half-life time.

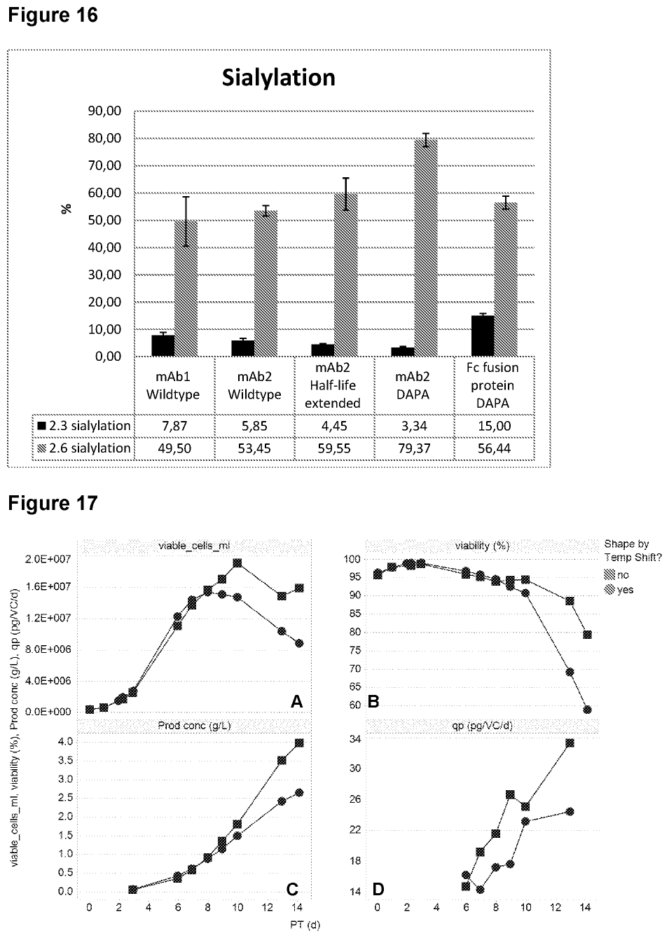

The capability of 2,6-sialylation of the best geCHO clone (stable transfected with the vector encoding ST6Gal-I (downstream of a CMV promoter), B4galt1 and Slc35a1 (downstream of SV40 promoter)) was further evaluated with a variety of different therapeutic protein formats up to pool level. The proteins which were evaluated are one Fc wildtype IgG antibody (mAb1), another IgG antibody (mAb2) with either Fc wildtype format, Fc half-life extension format or DAPA silencing format as well as an Fc fusion protein (DAPA format). Titers from fedbatch as well as sialylation level are shown in FIGS. 15 and 16.

The example of mAb2 shows that the Fc-structure also plays a role in the degree of 2,6-sialylation. The mAb2 WT showed the lowest degree of 2,6-sialylation (53% sialylation), the half-life extended form showed a slight increase of 2,6-sialylation (60% sialylation) and the DAPA form a significant increase of 2,6-sialylation (79% sialylation). The DAPA format contains key point mutations that abrogate binding of Fc receptors (FcγR, FcR) abolishing antibody directed cytotoxicity (ADCC) effector function. It is assumed that the DAPA mutation-set is somehow affecting the conformation of the Fc domain and therefore changes Fc glycosylation due to opening the Fc-structure around the N297 site. Enzymes such as e.g. galactosyltransferase or sialyltransferase might have better access to the N-linked glycosylation site. Also the half-life extended form with mutations in the constant domain CH2 might induce small conformational changes resulting in a more open “horseshoe”-Fc and better accessibility of enzymes to the glycosylation sites N297 and N297.

### Example 6: Cell Culture Process with the Glycoengineered CHO Cell Lines

A cell culture production process for geCHO cell line was developed to produce a highly sialylated Fc-multimer. The production process included the thawing of the cells in expansion medium (incl Puromycin and MTX) and splitting the cells two times in a 4:3:4 (4 day 3 day 4 day) rhythm before they were grown in the production bioreactor in a 10 L bench scale. The process conditions applied can be seen in table 1.

TABLE 1
Overview of production process conditions to investigate the
influence on growth, product concentration and sialylation
degree of geCHO cells expressing recombinant proteins.

| Process conditions | | 1 | 2 |
|---|---|---|---|
| Process duration [d] | | 14 | 14 |
| Cell line | | geCHO | geCHO |
| Cultivation medium | | Protein-free, chemically | Protein-free, chemically defined |
| | | defined production | production medium |
| | | medium | |
| Seeding VCD | | 4E5 cells/ml | 4E5 cells/ml |
| Temperature | | 36.5° C. | 36.5° C./T-shift to 33° C. |
| (set-point) | | | Start @ 12.5E6 ± 2.5E6 cells/ml |
| | | | latest 150 h post inoculation |
| Feeds | Feedrate 1 | 2% Feed 1 | 2% Feed 1 |
| | Feedrate 2 | 0.8% Feed 2 | 0.8% Feed 2 |

In FIG. 17 the growth and productivity data for the processes 1 and 2 are shown. With process condition 1 the cells grow up to 19e6 viable cells/mL, which is higher than with process condition 2 (maximum cell density 15e6 VCD/mL). After 14 days of cultivation the viability of the cells with process condition 1 are 20% higher than with process condition 2 (79% vs. 59% viability). The product concentration with process condition 1 (4 g/L) is 34% higher than with process condition 2 (2.65 g/L). The higher viable cell density and product concentration with process condition 1 results in a 36% higher specific productivity than with process condition 2 (33.34 pg/VC/d versus 24.44 pg/VC/d).

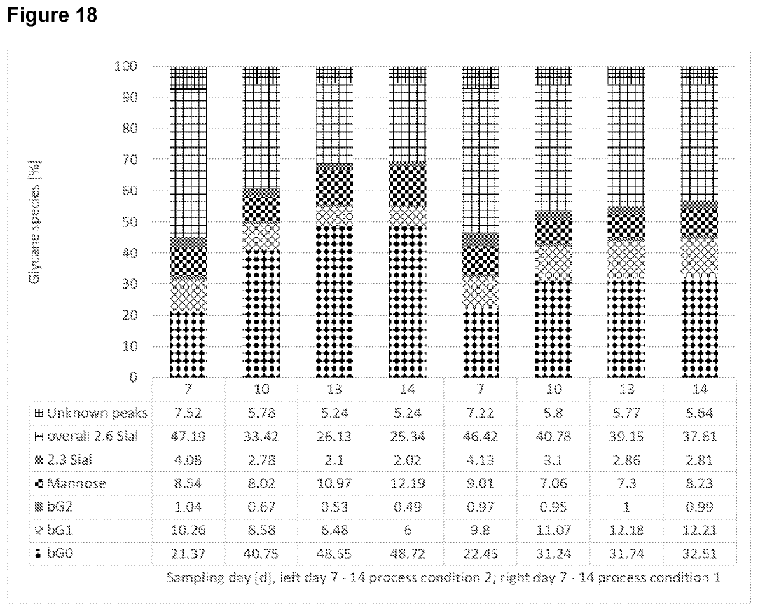

Besides growth and product formation the sialylation degree of the product is most important. In FIG. 18, the sialylation levels of the two tested process conditions are compared over time (days 7, 10, 13 and 14; condition 2 shown on the left, condition 1 shown on the right). The sialylation level was the same at day 7 (50%) with both process conditions. Toward day 14 the level of sialylation was surprisingly decreasing to 40.4% and 27.4% with process conditions 1 and 2 respectively. Therefore, with process condition 1 the sialylation level was much more stable and did not decrease as much as with process condition 2. The final sialylation degree with process condition 1 is 47% higher than the sialylation degree with process condition 2.

It was assumed that externally expressed sialidases or neuraminidases (Neu1, Neu2, Neu3 and Neu4, see Smutova et al. (2014) PLOS ONE 9(9): e106320) reduce the overall sialylation level of the expressed molecule. Therefore the following spike-in experiment was conducted:

Cells of parental geCHO cell line master cell bank were used to perform shake flask experiments (500 mL, 100 mL working volume, 200 rpm, 5% CO₂) in duplicate and process conditions 1 were applied (see table 1 above). To one set of shake flasks the polished material of the product (Fc-trimer) was added at day 0. To the other shake flask no product was added, which served as the reference.

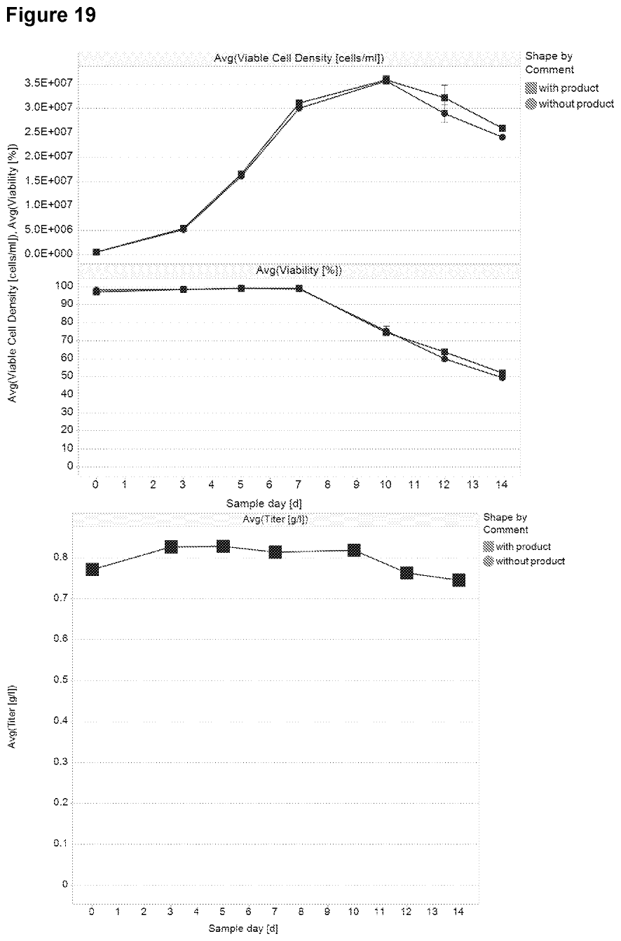

In FIG. 19 the viable cells, viability, and product concentration (titer) of the experimental setups is shown. No significant differences between both setups concerning viable cell density and viability were observable. The product concentration remains constant over the 14 days cultivation period, although the viability decline towards the end of the cultivation. Therefore, it can be concluded that the product does not have an influence on cell growth and viability.

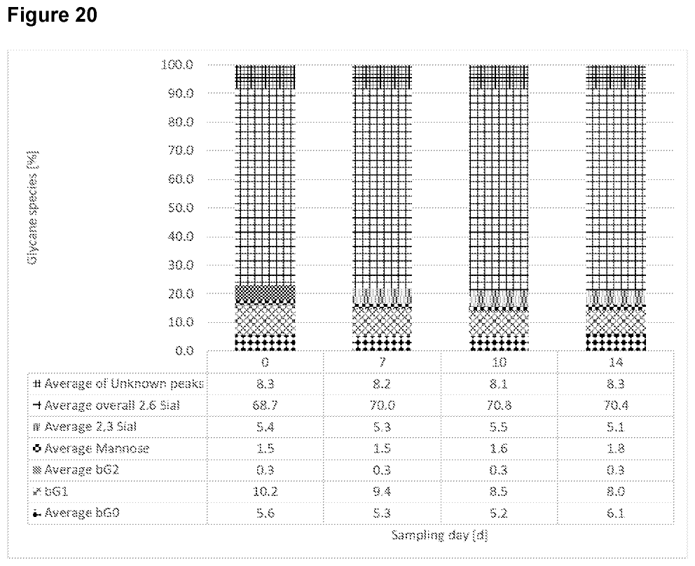

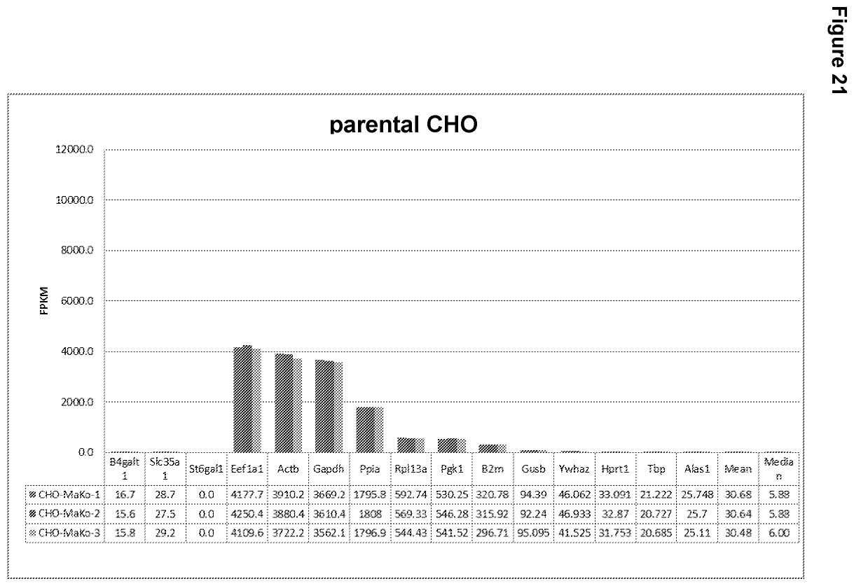

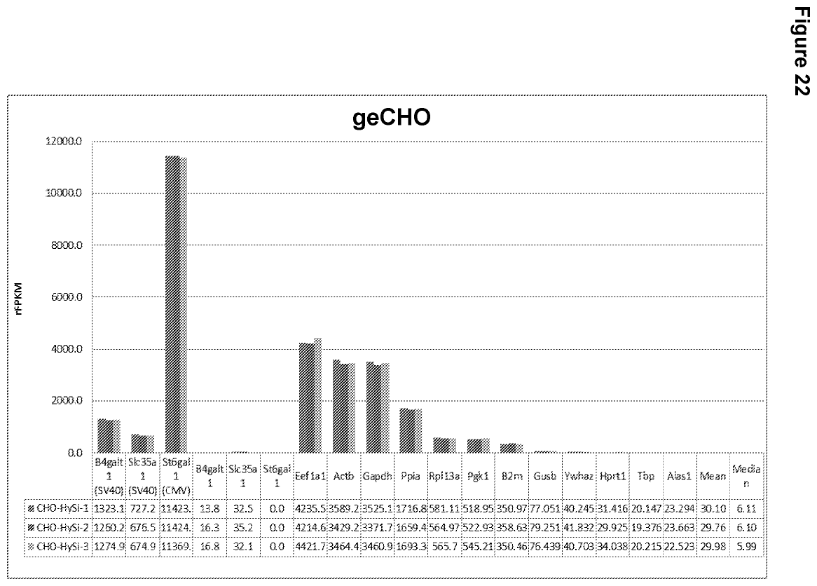

FIG. 20 shows the sialylation level of the product from shake flask one. At day 0, 7, 10 and 14 the 2.6-sialylation level is 68.7%, 70.0%, 70.8%, and 70.4% respectively, and therefore the 2.6-sialylation level does not change over time. It appears that the Neuraminidases do not have an influence on the sialylation pattern at these cultivation parameters.

### SUMMARY

By growing the cells at the constant temperature of 36.5° C. the cells grew to a higher cell number, the product concentration was higher and the sialylation level was surprisingly 47% higher compared to the same process where the cultivation temperature was shifted to 33° C. once the culture reached high cell density. Additionally, process condition 1 adds simplicity to the production process, since there is one process event less to consider (no shift in culture temperature). Therefore, the process is less likely to experience deviations and hence, is more robust.

It could also be shown that the decrease in sialylation level was not because of externally expressed sialidase activity in the medium. The product was surprisingly stable over the cultivation period.

### Example 7: Expression Level of the Introduced Glycosylation Enzymes

To determine the gene expression level of the exogenous expressed genes St6gal1 (downstream of CMV promoter), B4galt1 (downstream of SV40 promoter) and Slc35a1 (downstream of SV40 promoter) and therefore also the strength of the corresponding promoters next generation sequencing (transcriptomics) was performed. Sequencing libraries were prepared according to using Illumina's TruSeq Stranded Total RNA Sample Preparation with Ribo-Zero Gold and sequenced on a HiSeq 2500 with 76 bp reads in paired-end mode (2×76+8). The sequence reads were then aligned against the GCF_000223135.1_CriGri_1.0 reference genome using STAR (vers.2.5.2a), gene-level transcript counts were normalized to FPKM (Fragments Per Kilobase of transcript per Million mapped reads).

In FIGS. 21 and 22 the gene expression values of 12 representative endogenous expressed housekeeping genes are shown as FPKM. Additionally, the mean and median of all expressed genes (18,516 genes) are also shown. The gene expression level of the house keeping genes varied between ca. 20 FPKM and 4000 FPKM. The mean of all expressed genes was 30 FPKM and the median was around 6 FPKM. Additionally, the figures show also the expression values of the endogenous expressed St6gal1, B4galt1 and Slc35a1. The values shown in FIG. 21 are for parental CHO and in FIG. 22 for geCHO (selected clone for MCB).

The gene expression of these housekeeping genes as well as the mean and median values of the gene expression of all expressed genes was very similar for the geCHO cell line (which originates from the parental CHO cell line). Additionally, we have measured the gene expression of the exogenous expressed genes St6gal1, B4galt1 and Slc35a1. The highest expressed gene was St6gal1 downstream of the CMV promoter. The gene expression of exogenous B4galt1 and Slc35a1 (both downstream of SV40 promoter) were up to 16 fold lower compared to St6gal1 gene expression driven by CMV promoter. Overall the gene expression values of St6gal1 (downstream of the CMV promoter) were the highest among all expressed genes (almost 3 times higher expressed compared to the highest expressed housekeeping gene) highlighting that the CMV promoter is driving a very strong gene expression. In contrast, the genes B4galt1 and Slc35a1 downstream of the SV40 promoter were in a similar range as medium strong expressed housekeeping genes (FPKM values of around 300-1800). The gene expression values of the endogenous B4galt1 is 55 fold lower compared to the exogenous B4galt1 and the gene expression of the endogenous Slc35a1 was 15 fold lower compared to the exogenous Slc35a1. As expected endogenous St6gal1 was not expressed in CHO.

### Example 8: Recognition and Uptake of Hypersialylated Antibodies by Dendritic Cells

### FACS-Based Assay

Binding and internalization of a model antibody (mAbX1) produced in parental CHO (WT) or geCHO (HySi) was assessed on immature dendritic cells (IDCs) using a FACS-based assay. As further control, mAbX1 with a high amount of high-mannose type glycans (HiMan) and mAbX1 wherein the glycosylation site was removed by an N297A mutation (N297A) were used.

For the FACS-based internalization assay, IDCs (1.5×10⁵ per sample) were incubated with 10 μg/mL unlabeled mAbX1 glycovariants in binding buffer (HBS (Hepes buffered saline)+1 mM CaCl₂), 1 mM MgCl₂, 1 mM MnCl₂) at 4° C. (for binding) or 37° C. (for internalization) for 15, 30, 60 and 120 min. IDCs were washed to remove excess free mAbX1. Then, the residual amount of surface-bound mAbX1 was detected using 10 μg/mL FITC-labelled anti-mAbX1. After another wash step, stained IDCs were fixed (1% paraformaldehyde) and measured on the Attune NxT flow cytometer. Staining of antigen-positive Ramos cells was performed initially to confirm that the indirect staining protocol is valid and that mAbX1 glycovariants bind equally to the specific antigen.

The difference of the 4° and 37° C. fluorescence signal was calculated and scaled using the plogis( ) function in R and expressed as percentage of internalization.

Mannosylation of mAbX1 clearly increased the recognition and internalization (median internalization=84.6%) compared to the WT (median internalization=3.6%). Contrary, hypersialylation of mAbX1 (median internalization=0.2%) decreased the recognition and internalization compared to the WT (FIG. 24A).

Altered glycosylation did not affect Fab-mediated binding to the target antigen because all mAbX1 glycovariants showed equal binding to antigen-positive Ramos cells (FIG. 24B).

In summary, the data show that recognition by IDCs can be impacted by modifying antibody glycosylation, with high sialylation reducing recognition of the antibody by IDCs.

### Confocal Microscopy

Results from the FACS-based internalization assay indicate only residual mAbX1 cell surface binding. In order to prove that mAbX1 is taken up into the cell, binding and internalization of fluorochrome-labelled mAbX1 glycovariants by IDCs was assessed with confocal microscopy.

IDCs were seeded at 3×10⁵ cells in 300 μL differentiation medium per chamber of an 8 well chamber slide and incubated over night at 37° C. and 5% CO₂. On the next day, medium was replaced by binding buffer (HBS+1 mM CaCl₂), 1 mM MgCl₂) containing 20 g/mL AF647-conjugates of mAbX1 glycovariants and incubated for 120 min at 4° C. (fridge) and 37° C. and 5% CO₂. IDCs were fixed (4% paraformaldehyde) and permeabilized (0.1% Triton X-100 in PBS). After a blocking step with 2% BSA in PBS IDCs were incubated with marker antibodies for LAMP-1, EEA1 and Rab7 followed by the secondary donkey anti-rabbit-AF488. DAPI (4′,6-diamidino-2-phenylindole) was added as nuclear stain. Stained IDCs were mounted in FluoSafe reagent (Calbiochem) and covered with a glass slide. Cured slides were imaged on an Olympus FV3000 at 40× magnification. Quantification of internalized mAbX1 was done using HALO™ image analysis software (Akoya Biosciences).

Confocal images taken from IDCs incubated at 4° C. show that mAbX1 is solely localized at the cell surface characteristic of the expected ring structure of the fluorescence signal. At 37° C. mAbX1-derived fluorescence was detected in the cytoplasm adjacent to the nucleus (stained with DAPI) and not on the surface anymore.

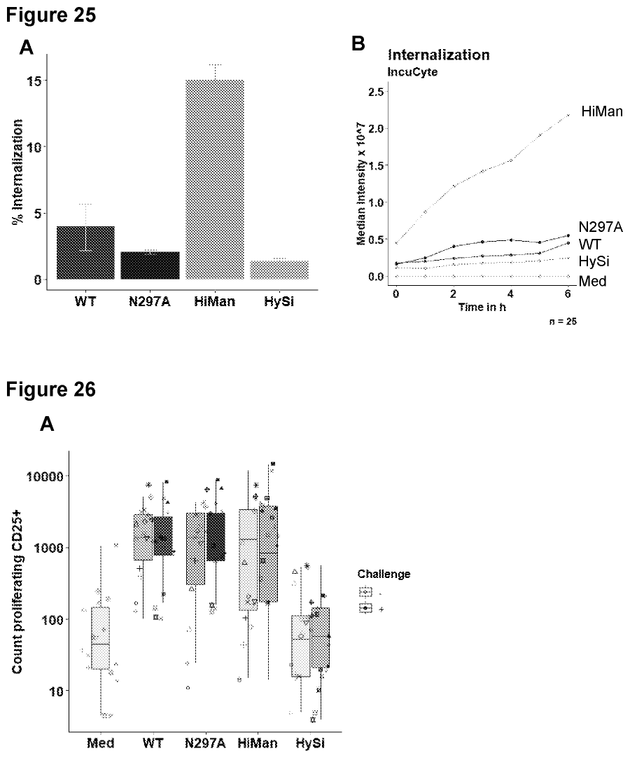

The glycosylation-dependent binding and internalization pattern of mAbX1 observed by FACS could be recapitulated: Aglycosylated mAbX1 revealed lower binding and internalization relative to the WT, mannosylated mAbX1 revealed strongest binding and internalization (FIG. 25A). Interestingly the hypersialylated mAbX1 showed a strong binding signal at 4° C. comparable to that of mannosylated mAbX1. However, at 37° C. only very weak internalization was detected for HySi. This observation suggests that the HySi is recognized by a different lectin receptor than the mannosylated mAbX1. This unknown receptor seems to cause a strong interaction but does not result in internalization of bound ligands and may therefore exert an inhibitory function.

Next, the effect of mAbX1 glycosylation on the endosomal routing was assessed. For this, IDCs were stained with the lysosomal marker LAMP-1 after internalization of fluorochrome-labelled mAbX1. mAbX1 was detected in lysosomal compartments and this effect was strongly associated with the glycosylation-pattern. Consistent with the strongest internalization of mannosylated mAbX1, this glycovariant caused most prominent routing into the lysosome. In contrast, hypersialylated mAbX1 demonstrated weak or no detectable co-localization with the lysosome. The N297A showed similar or slightly higher co-localization with LAMP-1. In addition to LAMP-1, EEA1 and Rab7 were used as markers to assess potential glycosylation-related differences in the routing into the early and late endosome, respectively. Clear co-localization with both markers was detected for HiMan, weak co-localization for WT and N297A with slightly stronger co-localization for N297A, and almost no detectable co-localization for HySi. At the tested time point (2 h) co-localization of mannosylated mAbX1 with the early endosome seemed to prevail. For the other glycovariants a difference in routing into either the early or late endosome could not be observed.

In summary, the data show that mAbX1 glycosylation determines the pattern of surface binding and intracellular uptake by IDCs. In addition, it was shown that hypersialylation decreases routing of mAbX1 into the degradative pathway.

### Real-Time Imaging

An additional method to determine internalization over time was established based on real-time imaging with the IncuCyte analyzer IncuCyte® Live cell Image Analyser (Sartorius). The principle is based on the detection of intracellular fluorescence signals resulting from internalization of fluorochrome-labeled mAbX1 glycovariants.

IDCs were seeded into a transparent flat bottom 96 well plate at 1×10⁵/well/100 UL and incubated over night at 37° C. and 5% CO₂. On the next day, the medium was replaced by medium containing 10 μg/mL AF647-conjugated mAbX1 glycovariant after the plate was kept for 10 min in the fridge. After 30 min incubation in the fridge, the supernatant was replaced by warm medium and placed immediately into the IncuCyte analyser. Images were acquired at 20× magnification every 20 min for the first 5 h and every hour for up to 24 h. Internalization was determined from quantification of intracellular fluorescence. Hereto, masks were created on phase and fluorescent objects using the Basic analyzer mode to capture IDCs and internalized mAbX1, respectively. Integrated Intensity (RCU×μm2) per well was reported.

Results obtained from the indirect FACS assay and the IncuCyte assay in the same donor were similar. Measuring the glycosylation-dependent internalization of mAbX1 by IDCs with the IncuCyte assay confirmed that mannosylation increased and hypersialylation decreased internalization at all measured time points (FIG. 25B).

Kinetic differences can be observed if the slopes for each glycovariants are compared. The slope is a measure to indicate the internalization rate per time unit. HiMan exerted the highest slope (0.273), followed by N297A (0.06) and WT (0.04). HySi revealed the lowest slope (0.02). Thus, HiMan shows the highest while HySi the lowest internalization rate/speed.

### Example 9: Recognition of Hypersialylated Antibodies by Human T Cells

The next question was whether glycosylation-mediated effects on internalization observed for mAbX1 implicate T cell activation. Therefore, internalization was investigated along with T cell activation in the same donor set (naive donors).

PBMCs (peripheral blood mononuclear cells) were isolated from buffy coats donated by naïve human subjects at blood donation center in Bern according to local ethical practices. Isolated PBMCs were stained with 5 μM CellTrace™ Violet (CTVio, LifeTech) for 20 min in a water bath (37° C.). Excess CTVio was removed after 5 min (RT) incubation of CTVio-incorporated cells with platelet-free autologous plasma by centrifugation at 360 g for 5 min. CTVio-negative PBMCs were used as control for compensation and as FMO (fluorescence minus one) control. CTVio+ PBMCs were seeded at 1×10⁶ cells/mL into 24 well plates in X-Vivo (Lonza)+5% platelet-free autologous plasma and stimulated (primed) with 1 and 10 μg/mL of the respective mAbX1 glycovariant, 5-30 μg/mL KLH (keyhole limpet hemocyanin, Thermo Scientific), 0.5 μg/mL Tetanus toxoid (TT, Enzo) or medium for 5 days. On day 5, DC-PBMC co-culture (1:10) was re-stimulated (challenged) with 1 and 10 μg/mL mAbX1 glycovariants, 5-30 μg/mL KLH, 0.5 μg/mL TT or medium and incubated for 4 additional days in presence of 5 U/mL IL-2. On day 9, stimulated cells were harvested and stained with the surface markers CD3, CD4, CD25, CD137 including a viability dye (Zombie aqua, Biolegend). Stained cells were measured on the Attune NxT flow cytometer using constant volumetric stop condition for all samples. FMO controls were used to discriminate between positive and negative population. Count of proliferating and activated Th cells were determined at priming and challenging and expressed as stimulation index (SI) indicating the challenge response relative to the priming response. Donors showing an SI above 1.5 were assigned as T cell responders.

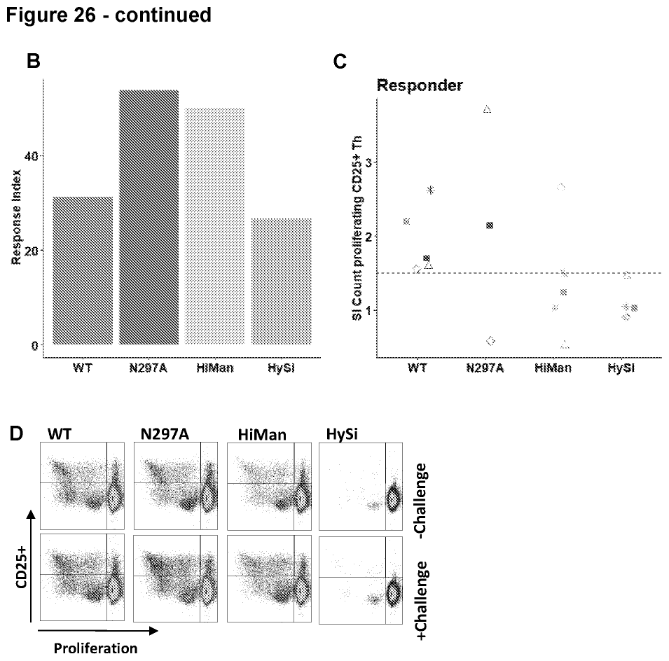

Five of 16 (31%) donors showed a WT-specific response (FIG. 26B). Mannosylated mAbX1 mounted an increased T cell response compared to WT with 7 responders of 16 tested donors (43%). Hypersialylated mAbX1 mounted a decreased T cell response compared to WT with 4 responders of 16 tested donors (25%). Of note, counts of proliferating CD25+ Th cells were remarkably lower if T cells were primed and challenged with HySi compared to all other mAbX1 glycovariants (FIG. 26A and FIG. 26D). Aglycosylated mAbX1 demonstrated similar or slightly higher T cell activation compared to mannosylated mAbX1 (47%). Donors were dissected in WT responder. None of the WT responder was reactive to hypersialylated mAbX1 (FIG. 26C), while few WT responders were reactive to HiMan and N297A. The positive control tetanus toxoid (TT) induced strong T cell activation (determined as CD25 upregulation) and proliferation indicating a good assay performance.

In summary, these data demonstrate that T cell responses can be enhanced or dampened if the glycosylation pattern is modified. While mannosylation enhances T cell responses compared to WT, hypersialylated mAbX1 is not recognized by WT responder T cells. This suggests that sialylation of mAbX1 does either cause less efficient antigen presentation, does not provide co-stimulatory signals to activate pre-existing WT-reactive T cells or/and promotes polarization to Tregs.

SEQUENCE LISTING

| SEQ | |
|---|---|
| ID NO: | Sequence |
| 1 | MIHTNLKKKFSYFILAFLLFALICVWKKGSYEALKLQAKEFQVTRSLEKLAMRSGSQS |
| | MSSSSKQDPKQDSQVLSHARVTAKVKPQPSYQVWDKNSSSKNLNPRLQKILKNYLNMN |
| | KYKVSYKGPGPGVKFSAEALRCRLRDRVNVSMIEATDFPFNTTEWAGYLPKENIRTKA |
| | GPWHRCAVVSSAGSLKSSQLGREIDNHDAVLRENGAPVANFQQDVGTKTTIRLMNSQL |
| | ITTEKQFLKDSLYSEGILIVWDPSLYHADIPSWYQKPDYNFFETYKSYRKLYPDQPFY |
| | ILRPQMPWELWDIIQEIAPDRIQPNPPSSGMLGIMIMMTLCDQVDIYEFLPSRRKTDV |
| | CYYHQKFFDSACTMGAYHPLLFEKNMVKQLNEGTDEDIYIFGKATLSGERTIHC |
| 2 | MIHTNLKKKFSCCVLVFLLFAVICVWKEKKKGSYYDSFKLQTKEFQVLKSLGKLAMGS |
| | DSQSVSSSSTQDPHRGRQTLGSLRGLAKAKPEASFQVWNKDSSSKNLIPRLQKIWKNY |
| | LSMNKYKVSYKGPGPGIKFSAEALRCHLRDHVNVSMVEVTDFPENTSEWEGYLPKESI |
| | RTKAGPWGRCAVVSSAGSLKSSQLGREIDDHDAVLRENGAPTANFQQDVGTKTTIRLM |
| | NSQLVTTEKRFLKDSLYNEGILIVWDPSVYHSDIPKWYQNPDYNFFNNYKTYRKLHPN |
| | QPFYILKPQMPWELWDILQEISPEEIQPNPPSSGMLGIIIMMTLCDQVDIYEFLPSKR |
| | KTDVCYYYQKFFDSACTMGAYHPLLYEKNLVKHLNQGTDEDIYLLGKATLPGFRTIHC |
| 3 | MRFLRPVLGGSAAMPGATLQRACRLLVAVCALHLGVTLVYYLSGRDLSRLPQLVGVSS |
| | TLRSGTIGATANKQPPGARPPPPVGVSSKPRPGPDSSPGTAFDPGLKSNWTSVLVPPT |
| | TALLTLPACPEESPLLVGPMVIDENIAVDLELLAKKNPEIKMGGRYSPKDCISPHKVA |
| | IIIPFRNRQEHLKYWLYYLHPVLQRQQLDYGIYVINQAGDTMFNRAKLLNIGFQEALK |
| | DHDYNCFVESDVDLIPMDDHNAYRCFSQPRHISVAMDKFGFSLPYVQYFGGVSALSKQ |
| | QFLAINGFPNNYWGWGGEDDDIFNRIVHKGMSISRPNAVVGRCRMIRHSRDKKNEPNP |
| | QRFDRIAHTKETMRFDGLNSLTYQVLNVERYPLYTKITVDIGTPR |
| 4 | MRLREPLLSGSAAMPGASLQRACRLLVAVCALHLGVTLVYYLAGRDLSRLPQLVGVST |
| | PLQGGSNSAAAIGQSSGELRTGGARPPPPLGASSQPRPGGDSSPVVDSGPGPASNLTS |
| | VPVPHTTALSLPACPEESPLLVGPMLIEFNMPVDLELVAKQNPNVKMGGRYAPRDCVS |
| | PHKVAIIIPFRNRQEHLKYWLYYLHPVLQRQQLDYGIYVINQAGDTIFNRAKLLNVGF |
| | QEALKDYDYTCFVFSDVDLIPMNDHNAYRCFSQPRHISVAMDKFGFSLPYVQYFGGVS |
| | ALSKQQFLTINGFPNNYWGWGGEDDDIFNRLVFRGMSISRPNAVVGRCRMIRHSRDKK |
| | NEPNPQRFDRIAHTKETMLSDGLNSLTYQVLDVQRYPLYTQITVDIGTPS |
| 5 | MAQARENVSLFFKLYCLAVMTLVAAAYTVALRYTRTTAKELYFSTTAVCVTEVIKLLI |
| | SVGLLAKETGSLGRFKASLSENVLGSPKELMKLSVPSLVYAVQNNMAFLALSNLDAAV |
| | YQVTYQLKIPCTALCTVLMLNRTLSKLQWVSVFMLCGGVILVQWKPAQATKVVVEQSP |
| | LLGFGAIAIAVLCSGFAGVYFEKVLKSSDTSLWVRNIQMYLSGIVVTLVGTYLSDGAE |
| | IKEKGFFYGYTYYVWFVIFLASVGGLYTSVVVKYTDNIMKGFSAAAAIVLSTIASVML |
| | FGLQITLSFAMGALLVCISIYLYGLPRQDTTCIQQEATSKERVIGV |
| 6 | MAAPRDNVTLLFKLYCLAVMTLMAAVYTIALRYTRTSDKELYFSTTAVCITEVIKLLL |
| | SVGILAKETGSLGRFKASLRENVLGSPKELLKLSVPSLVYAVQNNMAFLALSNLDAAV |
| | YQVTYQLKIPCTALCTVLMLNRTLSKLQWVSVEMLCAGVTLVQWKPAQATKVVVEQNP |
| | LLGFGAIAIAVLCSGFAGVYFEKVLKSSDTSLWVRNIQMYLSGIIVTLAGVYLSDGAE |
| | IKEKGFFYGYTYYVWFVIFLASVGGLYTSVVVKYTDNIMKGFSAAAAIVLSTIASVML |
| | FGLQITLTFALGTLLVCVSIYLYGLPRQDTTSIQQGETASKERVIGV |

1. A mammalian cell which is engineered for increased expression of an alpha-2,6-sialyltransferase, a beta-1,4-galactosyltransferase and a CMP-sialic acid transporter.

2. The mammalian cell according to claim 1, comprising
(i) an exogenous nucleic acid encoding an alpha-2,6-sialyltransferase;
(ii) an exogenous nucleic acid encoding a beta-1,4-galactosyltransferase; and
(iii) an exogenous nucleic acid encoding a CMP-sialic acid transporter.

3. The mammalian cell according to claim 2, comprising
(i) a first exogenous expression cassette comprising a first promoter operatively linked to a coding sequence for an alpha-2,6-sialyltransferase;
(ii) a second exogenous expression cassette comprising a second promoter operatively linked to a coding sequence for a beta-1,4-galactosyltransferase; and
(iii) a third exogenous expression cassette comprising a third promoter operatively linked to a coding sequence for a CMP-sialic acid transporter.

4. The mammalian cell according to claim 1, wherein endogenous genes of the mammalian cell encoding an alpha-2,6-sialyltransferase, a beta-1,4-galactosyltransferase and a CMP-sialic acid transporter are engineered for increased expression.

5. The mammalian cell according to claim 4, comprising
(i) a first exogenous promoter operatively linked to an endogenous coding sequence for an alpha-2,6-sialyltransferase;
(ii) a second exogenous promoter operatively linked to an endogenous coding sequence for a beta-1,4-galactosyltransferase;
(i) a third exogenous promoter operatively linked to an endogenous coding sequence for a CMP-sialic acid transporter.

6. The mammalian cell according to claim 3, wherein the first promoter is a strong promoter.

7. The mammalian cell according to claim 3, wherein the first promoter is a cytomegalovirus (CMV) promoter.

8. The mammalian cell according to claim 3, wherein the second and/or the third promoter is selected from the group consisting of simian virus 40 (SV40) promoter, CMV promoter, ubiquitin C (UBC) promoter, elongation factor 1 alpha (EF1A) promoter, phosphoglycerate kinase (PGK) promoter and β-actin promoter coupled with CMV early enhancer (CAGG), in particular SV40 promoter.

9. The mammalian cell according to claim 1, wherein the alpha-2,6-sialyltransferase is beta-galactoside alpha-2,6-sialyltransferase 1 (ST6GAL1), in particular derived from Cricetulus griseus or human; the beta-1,4-galactosyltransferase is beta-1,4-galactosyltransferase 1 (B4GALT1), in particular derived from Cricetulus griseus or human; and the CMP-sialic acid transporter is CMP-sialic acid transporter (SLC35A1), in particular derived from Cricetulus griseus or human.

10. The mammalian cell according to claim 1, wherein the mammalian cell is a rodent cell or human cell, in particular a Chinese hamster ovary (CHO) cell.

11. The mammalian cell according to claim 1, further comprising an exogenous expression cassette for recombinant expression of a glycosylated polypeptide.

12. A method for producing a glycosylated polypeptide, comprising the steps of
(a) providing a mammalian cell according to claim 11;
(b) cultivating the mammalian cell in a cell culture under conditions which allow for the expression of said glycosylated polypeptide;
(c) obtaining said glycosylated polypeptide from the cell culture; and
(d) optionally processing the glycosylated polypeptide.

13. The method according to claim 12, wherein the culture conditions during cultivation of the mammalian cell do not include a temperature shift of more than 2° C., in particular not a temperature shift of more than 1° C.

14. The method according to claim 12, wherein the temperature is kept within the range of 35 to 38° C. during cultivation of the mammalian cell.

15. The method according to claim 12, wherein step (d) comprises providing a pharmaceutical formulation comprising the glycosylated polypeptide.

16. The method according to claim 12, wherein the method is for producing a glycosylated polypeptide with reduced immunogenicity, and wherein the glycosylated polypeptide is a therapeutic antibody or a fragment, derivative or engraft thereof.

17. A vector nucleic acid or a combination of at least two vector nucleic acids, comprising
(i) a coding sequence for an alpha-2,6-sialyltransferase;
(ii) a coding sequence for a beta-1,4-galactosyltransferase; and
(iii) a coding sequence for a CMP-sialic acid transporter.

18. The vector nucleic acid or combination of at least two vector nucleic acids according to claim 17, comprising
(i) a first expression cassette comprising a first promoter operatively linked to the coding sequence for an alpha-2,6-sialyltransferase;
(ii) a second expression cassette comprising a second promoter operatively linked to the coding sequence for a beta-1,4-galactosyltransferase; and
(iii) a third expression cassette comprising a third promoter operatively linked to the coding sequence for a CMP-sialic acid transporter.

19. The vector nucleic acid or combination of at least two vector nucleic acids according to claim 18, wherein
(i) the first promoter is a cytomegalovirus promoter (CMV); and/or
(ii) the second and/or the third promoter is selected from the group consisting of simian virus 40 promoter (SV40), CMV promoter, ubiquitin C (UBC) promoter, elongation factor 1 alpha (EF1A) promoter, phosphoglycerate kinase (PGK) promoter and β-actin promoter coupled with CMV early enhancer (CAGG), in particular SV40 promoter.

20. The vector nucleic acid or combination of at least two vector nucleic acids according to claim 17, wherein the alpha-2,6-sialyltransferase is beta-galactoside alpha-2,6-sialyltransferase 1 (ST6GAL1), in particular derived from Cricetulus griseus or human; the beta-1,4-galactosyltransferase is beta-1,4-galactosyltransferase 1 (B4GALT1), in particular derived from Cricetulus griseus or human; and the CMP-sialic acid transporter is CMP-sialic acid transporter (SLC35A1), in particular derived from Cricetulus griseus or human.

21. Use of the vector nucleic acid or combination of at least two vector nucleic acids according to claim 17 for the transfection of a mammalian cell, in particular a Chinese hamster ovary (CHO) cell.

22. A method for increasing expression of an alpha-2,6-sialyltransferase, a beta-1,4-galactosyltransferase and a CMP-sialic acid transporter in a mammalian cell, comprising the step of transfecting the mammalian cell with the vector nucleic acid or a combination of at least two vector nucleic acids according to claim 17, and/or the step of engineering endogenous genes of the mammalian cell encoding an alpha-2,6-sialyltransferase, a beta-1,4-galactosyltransferase and a CMP-sialic acid transporter for increased expression.

23. A method for reducing the immunogenicity of an antibody or a fragment, derivative or engraft thereof, comprising the step of increasing the amount of sialylation in the glycosylation pattern of the antibody or fragment, derivative or engraft thereof.

24. The method according to claim 23, wherein the step of increasing the amount of sialylation includes one or more of
(i) treating the antibody or fragment, derivative or engraft thereof with a sialyltransferase and a sialic acid donor so that sialic acid residues are attached to the glycans present on the antibody or fragment, derivative or engraft thereof;
(ii) enriching those antibody or fragment, derivative or engraft thereof which carry at least one sialic acid;
(iii) producing the antibody or fragment, derivative or engraft thereof using a production method which results in a high amount of sialylation.

25. The method for reducing the immunogenicity of an antibody or a fragment, derivative or engraft thereof, comprising the step of increasing the amount of sialylation in the glycosylation pattern of the antibody or fragment, derivative or engraft thereof,
wherein the immunogenicity of the antibody or fragment, derivative or engraft thereof is reduced compared to a reference antibody or fragment, derivative or engraft thereof which has the same amino acid sequence and a relative amount of sialylation of 5% or less, and wherein the step of increasing the amount of sialylation includes producing the antibody or fragment, derivative or engraft thereof in the mammalian cell according to claim 1.

26. The method for reducing the immunogenicity of an antibody or a fragment, derivative or engraft thereof, comprising the step of increasing the amount of sialylation in the glycosylation pattern of the antibody or fragment, derivative or engraft thereof,
wherein the immunogenicity of the antibody or fragment, derivative or engraft thereof is reduced compared to a reference antibody or fragment, derivative or engraft thereof which has the same amino acid sequence and a relative amount of sialylation of 5% or less, and wherein the step of increasing the amount of sialylation includes producing the antibody or fragment, derivative or engraft thereof in the mammalian cell using a method according to claim 12.


SEQUENCE LISTING (6 sequences)

SEQ ID NO: 1 - protein, 402 aa, Cricetulus griseus
MIHTNLKKKFSYFILAFLLFALICVWKKGSYEALKLQAKEFQVTRSLEKLAMRSGSQSMSSSSKQDPKQDSQVLSHARVTAKVKPQPSYQVWDKNSSSKNLNPRLQKILKNYLNMNKYKVSYKGPGPGVKFSAEALRCRLRDRVNVSMIEATDFPFNTTEWAGYLPKENIRTKAGPWHRCAVVSSAGSLKSSQLGREIDNHDAVLRFNGAPVANFQQDVGTKTTIRLMNSQLITTEKQFLKDSLYSEGILIVWDPSLYHADIPSWYQKPDYNFFETYKSYRKLYPDQPFYILRPQMPWELWDIIQEIAPDRIQPNPPSSGMLGIMIMMTLCDQVDIYEFLPSRRKTDVCYYHQKFFDSACTMGAYHPLLFEKNMVKQLNEGTDEDIYIFGKATLSGFRTIHC

SEQ ID NO: 2 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 3 - protein, 393 aa, Cricetulus griseus
MRFLRPVLGGSAAMPGATLQRACRLLVAVCALHLGVTLVYYLSGRDLSRLPQLVGVSSTLRSGTIGATANKQPPGARPPPPVGVSSKPRPGPDSSPGTAFDPGLKSNWTSVLVPPTTALLTLPACPEESPLLVGPMVIDFNIAVDLELLAKKNPEIKMGGRYSPKDCISPHKVAIIIPFRNRQEHLKYWLYYLHPVLQRQQLDYGIYVINQAGDTMFNRAKLLNIGFQEALKDHDYNCFVFSDVDLIPMDDHNAYRCFSQPRHISVAMDKFGFSLPYVQYFGGVSALSKQQFLAINGFPNNYWGWGGEDDDIFNRIVHKGMSISRPNAVVGRCRMIRHSRDKKNEPNPQRFDRIAHTKETMRFDGLNSLTYQVLNVERYPLYTKITVDIGTPR

SEQ ID NO: 4 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 5 - protein, 336 aa, Cricetulus griseus
MAQARENVSLFFKLYCLAVMTLVAAAYTVALRYTRTTAKELYFSTTAVCVTEVIKLLISVGLLAKETGSLGRFKASLSENVLGSPKELMKLSVPSLVYAVQNNMAFLALSNLDAAVYQVTYQLKIPCTALCTVLMLNRTLSKLQWVSVFMLCGGVILVQWKPAQATKVVVEQSPLLGFGAIAIAVLCSGFAGVYFEKVLKSSDTSLWVRNIQMYLSGIVVTLVGTYLSDGAEIKEKGFFYGYTYYVWFVIFLASVGGLYTSVVVKYTDNIMKGFSAAAAIVLSTIASVMLFGLQITLSFAMGALLVCISIYLYGLPRQDTTCIQQEATSKERVIGV

SEQ ID NO: 6 - protein, 337 aa, Homo sapiens
MAAPRDNVTLLFKLYCLAVMTLMAAVYTIALRYTRTSDKELYFSTTAVCITEVIKLLLSVGILAKETGSLGRFKASLRENVLGSPKELLKLSVPSLVYAVQNNMAFLALSNLDAAVYQVTYQLKIPCTALCTVLMLNRTLSKLQWVSVFMLCAGVTLVQWKPAQATKVVVEQNPLLGFGAIAIAVLCSGFAGVYFEKVLKSSDTSLWVRNIQMYLSGIIVTLAGVYLSDGAEIKEKGFFYGYTYYVWFVIFLASVGGLYTSVVVKYTDNIMKGFSAAAAIVLSTIASVMLFGLQITLTFALGTLLVCVSIYLYGLPRQDTTSIQQGETASKERVIGV